(19) United States
(12) Patent Application Publication
(10) Pub. No.: US 2010/0234348 A1
(43) Pub. Date: Sep. 16, 2010

(54) COMPOSITIONS AND METHODS FOR POTENTIATING ANTIBIOTIC ACTIVITY

(75) Inventors: Guillaume Cottarel, Mountain View, CA (US); Timothy S. Gardner, Needham, MA (US); Xiaoguang Lei, Beijing (CN); John Porco, Jamaica Plain, MA (US); Scott E. Schaus, Boston, MA (US); Jamey Wierzbowski, Stoneham, MA (US); Kollol Pal, Needham, MA (US)

Correspondence Address: CHOATE, HALL & STEWART LLP, TWO INTERNATIONAL PLACE, BOSTON, MA, 02110, US

(73) Assignee: TRUSTEES OF BOSTON UNIVERSITY, Boston, MA (US)

(21) Appl. No.: 12/376,288

(22) PCT Filed: Aug. 2, 2007
(86) PCT No.: PCT/US07/75093

§ 371 Date: Jun. 1, 2010

Related U.S. Application Data
(60) Provisional application No. 60/835,710, filed on Aug. 4, 2006.

(51) Int. Cl. A61K 31/4035; C07D 405/08; C07D 487/04; C07D 498/06; C07C 69/00; C07D 231/56; A61K 31/5025; A61K 31/553; A61K 31/22; A61K 31/416; C12Q 1/18; C12Q 1/68; A61P 31/00

(52) U.S. Cl. 514/211.08; 548/454; 544/236; 540/545; 560/257; 548/362.5; 514/414; 514/248; 514/546; 514/403; 435/32; 435/6

(57) ABSTRACT

The present invention provides compounds that potentiate the activity of antibiotic agents, particularly quinolones such as norflaxin. The invention further provides compositions, e.g., pharmaceutical compositions, comprising the inventive compounds. The invention also provides compositions comprising an antibiotic (e.g., a quinolone) and a compound that potentiates activity of the antibiotic. The invention further provides methods of treating a subject comprising administering any of the inventive compounds or compositions to the subject. The invention also provides screening methods to identify compounds that potentiate the activity of an antibiotic, e.g., a quinolone.

### RELATED APPLICATIONS

This application claims priority from Provisional Application U.S. Ser. No. 60/835,710 filed on Aug. 4, 2006, which is incorporated herein by reference in its entirety.

### BACKGROUND OF THE INVENTION

The emergence of resistance to current antibacterial drugs is a growing problem for human health which also has enormous economic consequences (S. B. Levy, “The Antibiotic Paradox”, 1992, Plenum Press: New York, N.Y.). About 2 million people acquire bacterial infection in U.S. hospitals each year, and 90,000 die as a result. It has been shown that about 70% of those infections are resistant to at least one antibiotic. Resistant bacteria lead to higher health care cost because they often require more expensive drugs and extended hospital stays. The total cost to U.S. society is estimated at $30 billion annually. The trend towards increasing numbers of infection shows no sign of abating and the pace at which drug resistance increases is accelerating. Furthermore, infections due to drug-resistant bacteria are no longer confined to hospital and nursing home wards but are active in the community at large.

For decades, research and development efforts by large pharmaceutical companies have provided new drugs in time to combat rapidly evolving pathogens. While strategies for addressing antimicrobial drug resistance stress the urgent need for new drugs (Interagency Task Force on Antimicrobial Resistance, “Public health action plan to combat antimicrobial resistance”, Centers for Disease Control and Prevention, Atlanta, 2001; World Health Organization, “WHO global strategy for the containment of antimicrobial resistance”, Geneva, 2001; Commission of the European Council, “Community strategy against antimicrobial resistance”, Brussels, 2001; D. M. Livermore, Clin. Microbiol. Infect., 2004, 10: 1-9), recently, the flow of new antibiotics launched onto the market has dramatically decreased (J. H. Powers, Clin. Microbiol. Infect., 2004: 10: 23-31).

Efforts to overcome the growing problem of resistance have included modification of existing antibiotics, classical screening of new compound libraries and natural product libraries, and genomic efforts to identify novel targets to which no cross resistance with existing antibiotics would be anticipated. Even with this significant antibiotic discovery effort, only a few antibiotics have been approved by the U.S. Food and Drug Administration in recent years. In addition, few novel antibiotics that overcome resistance to current antibiotics are in clinical development. Particularly disturbing is that there is almost no antibiotic classes for which a bacterial resistance mechanism does not already exist. Most antibiotics in use today are chemically related to earlier ones discovered in the '40s, '50s and '60s; and only a very small number of new antibiotics with truly novel modes of action have been introduced during the last decade.

There is clearly a need in the art for new approaches to antibiotic drug discovery, for new antibiotics with improved properties, and for novel alternative strategies to antibiotic use.

### SUMMARY OF THE INVENTION

The invention encompasses the recognition that identification of compounds that potentiate the activity of known antibiotic substances would be of significant value both in terms of reducing the amount of such antibiotic need to treat or prevent an infection and in overcoming antibiotic resistance. In certain embodiments of the invention, the potentiating compound is active by itself as an antibiotic. In other embodiments of the invention, the potentiating compound is not active by itself as an antibiotic (at doses that can be tolerated according to sound medical practice).

In certain embodiments of the invention, including the potentiating compound in a therapeutic regimen comprising a first antibiotic agent allows a reduction in dose of said first antibiotic agent that is necessary to achieve a desired clinical effect.

In other embodiments of the invention, including the potentiating compound in a therapeutic regimen comprising a first antibiotic agent provides efficacy against an infectious agent that would otherwise be resistant to the first antibiotic agent.

In certain embodiments of the invention, including the potentiating compound in a therapeutic regimen allows an antibiotic that is otherwise cytostatic (i.e., prevents growth and multiplication of the infectious agent but does not kill it) to exert a cytotoxic effect (i.e., kills the infectious agent).

In certain embodiments of the invention, a combination of an antibiotic and the potentiating compound is therapeutically effective when delivered by a route of administration by which the antibiotic agent by itself would not be effective. For example, including the potentiating compound in a therapeutic regimen may allow delivery of the first antibiotic by the oral route rather than the intravenous route.

In certain embodiments of the invention, including the potentiating compound in a therapeutic regimen comprising a first antibiotic agent reduces the dosing interval of the first antibiotic needed to achieve a desired therapeutic effect. For example, the antibiotic agent may be effective for a longer period of time in the presence of the potentiating compound.

In certain embodiments of the invention, including the potentiating compound in a therapeutic regimen allows the use of an antibiotic that is highly potent but too toxic for the therapeutic use, i.e., the potentiating compound allows a lower dose of the antibiotic to be effective such that the antibiotic can be safely used without undue side effects.

Any and all of the above effects may be provided by the potentiating compound. Thus, a compound is said to “potentiate” an antibiotic if, for example, (i) in the presence of the compound, the concentration of antibiotic needed to achieve a given effect is lowered; and/or (ii) in the presence of the compound, the spectrum of infectious agents that can be treated by the antibiotic is expanded; and/or (iii) in the presence of the compound, an infectious agent that would otherwise be resistant to the antibiotic is sensitive to it.

The antibiotic potentiating compound may work by any of a variety of different mechanisms. It may or may not affect the same molecular target or pathway (e.g., metabolic pathway, biosynthetic pathway) as the antibiotic(s) whose activity it potentiates. The potentiating compound may inhibit metabolism of the antibiotic. For example, the potentiating agent may be an inhibitor (e.g., a competitive or non-competitive inhibitor) of an enzyme that degrades the antibiotic. The potentiating compound may alter the mechanism of the antibiotic, e.g., may increase metabolism to a more active form, shift the profile of metabolites, etc. The potentiating compound may alter distribution, absorption, or excretion of the antibiotic in a way that effectively increases its activity in the body. The potentiating compound and the antibiotic may exhibit a “synthetic lethal” effect, i.e., the combination of the two is lethal at concentration at which neither is lethal by itself. Preferably, the compound is substantially non-toxic to mammalian cells over a wide range of concentrations, including concentrations at which it effectively potentiates the activity of an antibiotic.

The antibiotic can belong to any class of antibiotic agents in various embodiments of the invention. The antibiotic can be a broad spectrum or narrow spectrum agent. It may be active against Gram positive bacteria, Gram negative bacteria, or both. It may be active against acid fast bacilli. It may be active against species of infectious agent.

The invention also encompasses the recognition that identification of the molecular target of such antibiotic potentiating compound provides a means of identifying new antibiotics that act on these agents. The new antibiotics may be active in the absence of other antibiotic agents and/or potentiate the effect of other antibiotic agents.

Certain antibiotics are believed to act by inhibiting bacterial DNA gyrase, topoisomerase IV, or both. Among these antibiotic classes are the quinolones. DNA gyrase inhibitors, particularly quinolones, serve as an exemplary class of antibiotic agents with which to put these approaches into effect.

In one aspect, the present invention provides novel compounds that potentiate the effects of an antibiotic that has activity as a DNA gyrase inhibitor, a topoisomerase inhibitor (e.g., a topoisomerase IV inhibitor), or both. Preferably, the DNA gyrase or topoisomerase is a DNA gyrase or topoisomerase that is found in a bacteria, fungus, protozoa, or other parasite. In certain embodiments of the invention, the antibiotic is a quinolone antibiotic, e.g., a fluoroquinolone. In certain embodiments of the invention, the quinolone is norfloxacin. In other embodiments of the invention, the quinolone is selected from the group consisting of, but not limited to, ciprofloxacin, ofloxacin, levofloxacin. In certain embodiments of the invention, the potentiating compound has a flavone backbone structure. In some embodiments, the flavone comprises a halogen atom, e.g., bromine in certain embodiments of the invention, the potentiating compound is one of compounds 1-10, as shown in FIG. 1, identified by names X1-flavo-1 through X1-flavo-10. In other embodiments of the invention, the potentiating compound has a terpene backbone structure, e.g., a triterpene structure. In some embodiments, the potentiating compound is the compound shown in FIG. 2 (i.e., CB101). It will be appreciated that a variety of compounds having the same core structure can be synthesized comprising any of a plurality of different functional groups. Such compounds will likely include additional antibiotic potentiating compounds (e.g., compounds that potentiate quinolone activity). Certain of the compounds may display higher potentiating activity that the compounds depicted in FIGS. 1 and 2. For example, such compounds may be any of the compounds shown in FIGS. 45, 46(A) and 46(B), and 48 and derivatives thereof. In some embodiments, the potentiating compound is the compound shown in FIG. 3. In some embodiments, the potentiating compound is the compound shown in FIG. 39 (i.e., CB201). Any of the compounds of the invention may be provided in isolated or purified form.

In another aspect, the present invention provides compositions comprising the inventive potentiating compounds, e.g., compositions comprising a pharmaceutically acceptable carrier, diluent, excipient, etc.

The present invention also provides compositions comprising one or more of the inventive compounds and an antibiotic whose activity is potentiated by one or more of the inventive compounds.

In still another aspect, the present invention further provides a method of treating a subject in need thereof comprising the step of administering any of the inventive compounds or compositions thereof to the subject. Preferably, the potentiating compounds are administered to a subject who is also receiving the antibiotic whose activity the compound potentiates. The antibiotic and the potentiating compound may be administered concurrently or sequentially. They may be administered together in a single composition or separately. They may be delivered by the same route of administration or different routes.

The present invention further provides a method of identifying an antibiotic potentiating compound comprising the steps of: contacting a cell with an antibiotic agent and a candidate compound; and identifying the compound as an antibiotic potentiating compound If the growth of the cell in the presence of the antibiotic agent and the compound is less than the growth of the cell in the presence of the antibiotic agent alone under similar conditions (e.g., similar concentration, temperature, etc). Preferably, the cell is a bacterial cell, fungal cell or protozoal cell. The cells can be cultured in any convenient manner for performance of the screen. In certain embodiments of the invention, the screen is performed using a high-throughput format, e.g., using microwell plates.

The cell may be resistant to the antibacterial agent. The cell may be a mutant, e.g., it may have a deletion or inactivating mutation in or more genes, e.g., a gene that is postulated to be a target or receptor for an antibiotic or antibiotic potentiating compound. The cell may over-express one or more genes, e.g., a gene that is postulated to be a target for an antibiotic or antibiotic potentiating compound. Expression of the gene may be under control of an inducible or constitutive promoter. The cell may be temperature sensitive. In certain embodiments of the invention, the cell contains a plasmid, e.g., an expression vector.

In addition to screens performed on intact cells, the invention also encompasses cell-free screening assays using, for example, in vitro systems that recapitulate important pathways or enzymatic activities of an infectious agent. For example, the screen can identify compounds that potentiate the inhibitory effect of an antibiotic on an enzymatic activity.

In certain embodiments of the invention, the compounds to be tested are synthesized to contain a common core structure, e.g., a flavone or terpene structure. In some embodiments of the invention, the compounds to be tested are any or all of around 1,200 compounds of the CMLD library developed by Boston University. In other embodiments of the invention, the compound to be tested are any or all of the compounds shown on FIGS. 45, 46(A) and 46(B), and 48 and derivatives thereof. In some embodiments, the compound to be tested is a flavone, a coumarin or a heterocyclic compound.

In a further aspect, the present invention provides a computer-readable medium on which are stored results of a screen to identify a compound that potentiates activity of an antibiotic. The results may be stored in a database and can include any screening protocols, results obtained from the screen or from additional screens, and/or protocols of or results obtained from tests performed on compounds identified in the screen (e.g., tests in animal models of infection).

In yet a further aspect, the present invention provides a method of conducting a business to identify a therapeutic agent, i.e., a compound that potentiates an antibiotic. The method involves performing any of the screens described herein, optionally to identify a compound that potentiates the activity of a marketed antibiotic, a non-marketed agent known to have antibiotic activity, etc. The screens can be performed on a contract basis, e.g., as a service, in which a customer requests on a contract basis, e.g., as a service, in which the customer requests that a screen be performed to identify a compound that potentiates activity of an agent suggested by or provided by the customer.

Those of skill in the art will appreciate that many of the compounds encompassed by the structures shown herein may exhibit the phenomena of tautomerism, conformational isomerism, geometric isomerism and/or stereoisomerism. As the formulae drawings within this specification can represent only one of the possible tautomeric, conformational isomeric, enantiomeric or geometric isomeric forms, it should be understood that the invention encompasses any tautomeric, conformation isomeric, enantiomeric and/or geometric isomeric forms of the compounds having one or more of the utilities described herein.

In addition, those of skill in the art will also recognize that the compounds of the invention may exist in many different protonation states, depending on, among other things, the pH of their environment. While the structural formulae provided herein depict the compounds in only one of several possible protonation states, it will be understood that these structures are illustrative only, and that the invention is not limited to any particular protonation state—any and all protonated forms of the compounds are intended to fall within the scope of the invention.

The compounds of the present invention may, in certain embodiments, bear one or more positive or negative charges and may have appropriate counter ions associated herewith. The net charge of the compound may be positive or negative. The identity of the associated counter ions may be governed by the synthesis and/or isolation methods by which the compounds are obtained. Counter ions include, but are not limited to, chloride and other halides, acetate, trifluoroacetate, citrate, sulfate, phosphate, sodium, magnesium, etc., and mixtures thereof. It will be understood that the identity of any associated counter ion is not a critical feature of the invention, and that the invention encompasses the compounds in association with any type of counter ion. Moreover, as the compounds can exist in a variety of different forms, the invention is intended to encompass not only forms that are in association with counter ions (e.g., dry salts), but also forms that are not in association with counter ions (e.g., aqueous or organic solutions).

Unless otherwise indicated, the present invention utilizes well-known methods of molecular biology, cell culture, etc., as described in, for example, “Current Protocols in Molecular Biology, and Current Protocols in Cell Biology”, John Wiley & Sons, N.Y., edition of July 2002; Sambrook et al., Molecular Cloning: A Laboratory Manual, 3^{rd} Ed., Cold Spring Harbor Laboratory Press, Cold Spring Harbor, 2001; N. Woodford and A. Johnson, “Molecular Bacteriology: Protocols and Clinical Applications”, Humana, 1998; Gerhardt et al., “Methods for General and Molecular Microbiology”, American Society for Microbiology, 1994, each of which is incorporated herein by reference.

### BRIEF DESCRIPTION OF THE DRAWING

FIG. 1 shows the structures of 10 compound that potentiate the activity of norfloxacin. The compounds are identified by plate, location, and name. The numbers at the left, below each compound, are the formula numbers referred to in the claims and elsewhere herein.

FIG. 2 shows the structure of CB101, which was identified as a compound that potentiates activity of norfloxacin.

FIG. 3 shows the structure of another compound that potentiates the activity of norfloxacin.

FIG. 4 shows growth curves demonstrating the ability of compound CB101 to potentiate the activity of norfloxacin. The curves show a dramatic reduction in bacterial growth in the presence of norfloxacin when the inventive compound is also present.

FIGS. 5-36 show diagrams of various aspects of the invention including screening strategies and methods of target identification and other information.

FIG. 37 is a scheme showing the synthetic steps used in the preparation of CB101.

FIG. 38 is a scheme showing how some of the compounds of the present invention, in particular, CB101 and CB201 have been identified.

FIG. 39 shows the structure of CB201, which was identified as a compound that potentiates activity of quinonoles.

FIG. 40 shows results of experiments carried out to test the effects of CB101 (FIG. 40(A)) and CB201 (FIG. 40(B)) on their potentiating activity in S. aureus (see Example 4).

FIG. 41 shows results of experiments carried out to test the ability of CB101 (FIG. 41(A)) and CB201 (FIG. 41(B)) to combat Staphylococcus clinical isolates resistant to ciprofloxacin (see Example 5).

FIG. 42 shows results of experiments carried out to validate the potentiating activity of CB101 in vivo. The in vivo system used in these experiments was mice infected with moderately fluoroquinolone resistant S7 Staphylococcus isolate (see Example 6).

FIG. 43 is a table summarizing the results of experiments carried out to validate the potentiating activity of CB101 in vivo. The in vivo system used in these experiments was mice infected with moderately fluoroquinolone resistant S7 Staphylococcus isolate (see Example 6).

FIG. 44 shows results of experiments carried out to test the effect of serum on the quinolone potentiating activity of CB101 and CB201 (see Example 7).

FIG. 45 shows the flavone (21150S) and the coumarin (19-281), which were found to exhibit quinolone potentiating activity (see Example 8).

FIG. 46 (part (A) and part (B)) shows heterocyclic analogs, which were found to exhibit quinolone potentiating activity (see Example 8).

FIG. 47 shows the structure of three intermediates involved in the synthesis of CB101 (see Example 9).

FIG. 48 shows the three libraries (library A, library B and library C) built on the three intermediates involved in the synthesis of CB101 (see Example 9).

### DEFINITIONS

Throughout the specification, several terms are employed that are defined in the following paragraphs.

The terms “antibiotic”, “antibiotic agent” and “antimicrobial agent” are used herein interchangeably. They refer to an agent that inhibits and/or stops growth and/or proliferation of one or more species of microorganisms (e.g., virus, bacteria, fungus, protozoa, helminth, fluke or other parasite). The antibiotic may display inhibitory activity in vitro (e.g., when contacted with cells in cell culture), in vivo (e.g., when administered to a subject at risk of or suffering from an infection), or both. The terms include bacteridical and bacteriostatic agents. The term “bactericidal”, when used herein in reference to an antibiotic agent, refers to an agent that kills bacteria. A bactericidal agent may inhibit or stop growth or proliferation of the bacteria before killing them. The term “bacteriostatic”, when used herein in reference to an antibiotic agent, refers to an agent that substantially inhibits or stops growth or proliferation of bacteria but does not kill them.

“Microbe”, “microbial” and like terms, as used herein, refer to microscopic organisms (e.g., bacteria or fungi). When used in reference to quinolone antibiotics, “microbe” and like terms typically refer to bacteria, although they can encompass any microorganism against which quinolone antibiotics display inhibitory activity.

A “strain” is a generic variant or subtype of a type or species of microorganism, e.g., an isolate of a microorganism that possesses the major properties that define the species or type but differs from many or most other members of the species or type in one or more other properties. The term “strain” can refer to a bacterium that harbors a particular episome or contains a particular mutation in a gene that is not found in many other subtypes or strains of the species.

The term “microbial infection” refers to the invasion of the host organism, whether the organism is a vertebrate, invertebrate, fish, plant, bird, or mammal, by pathogenic microbes (e.g., bacteria). This includes the excessive growth of microbes that are normally present in or on the body of a mammal or other organism. More generally, a microbial infection can be any situation in which the presence of a microbial population(s) is damaging to a host organism. Thus, an organism is “suffering from” a microbial infection when excessive numbers of a microbial population are present in or on the organism's body, or when the effects of the presence of a microbial population(s) is damaging to the cells or other tissue of an organism. The compounds and compositions of certain embodiments of the present invention are also useful in treating microbial growth or contamination of cell cultures or other media, or inanimate surfaces or objects, and nothing herein should limit the invention to treatment of higher organisms, except when explicitly so specified in the claims.

As used herein, the term “growth” refers to an increase in microbial biomass. The term “proliferation”, as used herein, refers to an increase in microbial number. Since bacterial proliferation is usually of primary concern, and since under most circumstances of interest herein proliferation is accompanied by an increase in microbial mass, the term “growth” is generally understood to mean “proliferation”, and the two terms are used interchangeably herein although it is recognized that difference assays may measure either or both of these parameters. For example, optical density reflects biomass and does not specifically reflect cell number, whereas an assay based on detecting colonies formed from individual cells reflects cell number rather than biomass.

The terms “minimal inhibitory concentration” (MIC) and “minimal bactericidal concentration” (MBC) are used herein consistently with their use in the art, i.e., to indicate the concentration of an agent that will inhibit bacterial proliferation (growth) (MIC) or kill bacteria (MBC). MIC values may be, for example, the concentration of agent that inhibits visible growth or may be expressed as MIC₅₀, MIC₉₀ or MIC₉₉ values, i.e., the concentration of an agent that reduces bacterial proliferation to 50% or less, 10% or less, or 1% or less, respectively, of the control value that would occur in the absence of the agent. As is well known in the art, MIC and MBC can be measured by a variety of methods, including automated and non-automated methods. Suitable methods are described in publications of the Clinical Laboratory Standards (CLSI), formerly the National Committee for Clinical Laboratory Standards (NCCLS).

To “potentiate” an agent means to enhance or increase at least one biological effect or activity of the agent so that either (i) a given concentration or amount of the agent results in a greater biological effect or activity when the agent is potentiated than the biological effect or activity that would result from the same concentration or amount of the agent when not potentiated; or (ii) a lower concentration or amount of the agent is required to achieve a particular biological effect or activity when the agent is potentiated than when the agent is not potentiated; or (iii) both (i) and (ii). The biological effect or activity may be, for example, the ability to catalyze or inhibit one or more chemical reactions, the ability to catalyze or activate one or more chemical reactions, the ability to activate or inhibit a biological or biochemical pathway, the ability to reduce or inhibit microbial proliferation, the ability to kill a microorganism, etc. A compound whose presence potentiates an active agent is referred to as a “potentiating compound”.

The term “in combination” as used herein with respect to administration of first and second agents is administration performed such that (i) a dose of the second agent is administered before more than 90% of the most recently administered dose of the first agent has been metabolized to an inactive form or excreted from the body; or (ii) doses of the first and second agents are administered within 48 hours of each other, or (iii) the agents are administered during overlapping time periods (e.g., by continuous or intermittent infusion); or (iv) any combination of the foregoing. The agents may, but need not be, administered together as components of a single composition. The agents may be administered individually at substantially the same time (by which is meant within less than 10 minutes of one another). The agents may be administered individually within a short time of one another (by which is meant less than 1 hour apart). The agents may, but need not, be administered by the same route of administration. When administered in combination with a second agent, the effective concentration of a first agent needed to elicit a particular biological response may be less than the effective concentration of the first agent when administered in the absence of the second agent, thereby allowing a reduction in the dose of the first agent relative to the dose that would be needed if the first agent was administered in the absence of the second agent. The effects of multiple agents may, but need not be, additive or synergistic. The agents may be administered multiple times.

The terms “local administration” and “local delivery” are used herein interchangeably. They refer to an administration/delivery that does not rely upon transport of an active agent to its intended target tissue via the vascular system. The agent is delivered directly to its intended target tissue or in the vicinity thereof, e.g., by injection or implantation.

As used herein, the term “subject” refers to an individual to whom an agent is to be delivered, e.g., for experimental, diagnostic, and/or therapeutic purposes. Preferred subjects are mammals, particularly domesticated mammals (e.g., dogs, cats, etc.), primates, and humans.

The term “effective amount”, as used herein with respect to an active agent, refers to the amount of active agent sufficient to elicit a desired biological response. As will be appreciated by those of ordinary skill in the art, the absolute amount of a particular agent that is effective may vary depending on such factors as the desired biological endpoint, the agent to be delivered, the target tissue, etc. Those of ordinary skill in the art will further understand that an “effective amount” may be administered in a single dose, or may be achieved by administration of multiple doses. For example, an effective amount may be an amount sufficient to achieve one or more of the following: (i) prevent or reduce the severity of one or more symptoms or signs of an infection; (ii) cause a reduction in number of infectious agents in a subject; (iii) prevent recurrence of an infection; (iv) prevent occurrence of a clinically significant infection in a subject who has been exposed to an infectious agent, etc.

The term “biological system”, as used herein, refers to any system containing at least one biological component, e.g., a biological macromolecule such as a protein or nucleic acid, suitable for performing an assay of a biological or biochemical function of activity. The term includes cell-free systems, cells, collections of cells, biological fluids, animals, etc.

As used herein, the term “high throughput screening” refers to an assay that allows for multiple candidate agents or samples to be screened substantially simultaneously. Such assays typically entail the use of microtiter (microwell) plate (e.g., plates having 96, 384 or 1596 wells) which are particularly convenient because a large number of assays can be carried out simultaneously, using small amounts of reagents and samples. Such assays may also advantageously minimize the number of steps such as washing cells, removing culture medium, and/or pipetting reagents.

The term “quinolone antibiotic” refers to an agent containing a quinolone or a naphthyridine nucleus with any of a variety of different side chains and substituents as known and understood in the art and that displays inhibitory activity towards one or more microbial species, e.g., various quinolinecarboxylic acids. The term “quinolone antibiotic” encompasses isolated enantiomers, salts, hydrates, or the free base form of any quinolone antibiotic.

As used herein, the term “isolated” means (i) separated from at least some of the components with which it is usually associated in nature; (ii) prepared or purified by a process that involves the hand of man; and/or (iii) not occurring in nature.

As used herein, the term “purified”, means separated from other compounds or entities. A compound or entity may be partially purified, substantially purified, or pure. A compound or entity is considered pure when it is removed from substantially all other compounds or entities, i.e., is preferably at least about 90%, more preferably at least 91%, 92%, 93%, 94%, 95%, 96%, 97%, 98%, 99%, or greater than 99% pure. A partially or substantially purified compound or entity may be removed from at least 50%, at least 60%, at least 70%, or at least 80% of the material with which it is naturally found, e.g., cellular material such as cellular proteins and/or nucleic acids, or with which it is found after synthesis, e.g., starting material(s), intermediate(s), and side-product(s).

As used herein, the term “small molecule” refers to organic compounds, whether naturally-occurring or artificially created (e.g., via chemical synthesis) that have relatively low molecular weight and that are not proteins, polypeptides, or nucleic acids. Typically, small molecules have a molecular weight of less than 1500 Daltons.

The term “treatment” is used herein to characterize a method that is aimed at: (i) delaying or preventing the onset of a medical condition, disease or disorder; (ii) slowing down or stopping the progression, aggravation, or deterioration of the symptoms of the condition; (iii) bringing about amelioration of the symptoms of the condition; and/or (iv) curing the condition. A treatment may be administered prior to the onset of the condition, for a prophylactic or preventive action, or it may be administered after initiation of the condition, for a therapeutic action.

A “pharmaceutical composition” is herein defined as comprising an effective amount of a potentiating compound and at least one physiologically acceptable carrier or excipient. A pharmaceutical composition can further comprise one or more antibiotics, e.g., one or more antibiotics whose action is known to be potentiated by the potentiating compound. Alternatively or additionally, a pharmaceutical composition can further comprise one or more additional therapeutic agents.

As used herein, the term “physiologically acceptable carrier or excipient” refers to a carrier medium or an excipient which does not interfere with the effectiveness of the biological activity of the active ingredient(s) of the composition and which is not excessively toxic to the host at the concentrations at which it is administered. The term includes solvents, dispersion media, coatings, antibacterial agents, isotonic agents, absorption delaying agents, and the like. The use of such media and agents for the formulation of pharmaceutically active substances is well known in the art (see, for example, Remington's Pharmaceutical Sciences”, E. W. Martin, 18^{th} Ed., 1990, Mack Publishing Co.: Easton, Pa., which is incorporated herein by reference in its entirety).

The term “comprising” is used herein in a general sense. It should be understood that, in general, where the invention, or aspects of the invention, is/are referred to as comprising particular elements, features, etc, certain embodiments of the invention or aspects of the invention consist, or consist essentially of, such elements, features, etc. For purposes of simplicity those embodiments have not been specifically set forth haec verba herein. Where ranges are given, endpoints are included.

“Liposomes” are artificial microscopic spherical particles in an aqueous medium, formed by a lipid bilayer (or multilayers) enclosing an aqueous compartment. Liposomes are commonly used as a delivery vehicle for various types of molecules (such as proteins, small molecules, DNA, and RNA), including a number of different drugs and can be used for delivering the compounds or compositions of the invention.

### DETAILED DESCRIPTION OF CERTAIN PREFERRED EMBODIMENTS

The present invention relates to the identification, production, and/or use of agents that can be useful in antibiotic therapy. In particular, the invention relates to the identification, production and/or use of compounds that potentiate the activity of antibiotic agents.

### I. Identification of Potentiating Compounds

In one aspect, the invention relates to screening methods for identifying compounds that potentiate the activity of an antibiotic agent. In particular, a method is provided that comprises steps of: contacting a cell with an antibiotic agent and a candidate compound, and identifying the candidate compound as an antibiotic potentiating compound if the growth of the cell in the presence of the antibiotic agent and the compound is less than the growth of the cell in the presence of the antibiotic agent alone under substantially equivalent conditions.

### A—Biological Systems

As will be appreciated by one of ordinary skill in the art, the assays and screening methods of the present invention can be performed using any type of biological systems, including cell-free systems (e.g., in vitro systems that recapitulate important pathways or enzymatic activities of an infectious agent), cells, collections of cells, biological fluids (e.g., blood sample, urine, synovial fluid, etc, infected with the infectious agent), or animals (e.g., animal models of particular infections).

The cell-based screening methods of the present invention may be carried out using any cell types that can be grown in standard tissue culture plastic ware. Such cells include all normal and transformed cells derived from any recognized sources. Preferably, cells are bacterial cells, fungal cells, or protozoal cells. Cells may be obtained by techniques well know in the art (for example, cells may be isolated from samples such as blood, urine, sputum, synovial fluid, cerebrospinal fluid, pus, or any sample of body fluid or tissue obtained from an individual suspected or diagnosed to be the host of a microorganism). Alternatively, cells may be purchased from immunological and microbiological commercial resources (for example, from the American Type Culture Collection, Manassas, Va.).

In certain embodiments, the cells used in the inventive screening methods comprise a heterogeneous population of cells (i.e., contains cells of more than one cell type). In other embodiments, the cells are of a single cell type. For example, cells are from a substantially homogeneous population of cells, wherein at least about 80%, and preferably at least about 90% of the cells in the population are of the same cell type.

Cells to be used in the inventive assays may be cultured according to standard cell culture techniques. For example, cells are grown in a suitable vessel in a sterile environment at 37° C. in an incubator or warm room in an appropriate cell culture medium. Vessels may contain stirred or stationary cultures. Cell culture techniques are well known in the art and established protocols are available for the culture of diverse cell types (N. Woodford and A. Johnson, “Molecular Bacteriology: Protocols and Clinical Applications”, Humana, 1998; Gerhardt et al., “Methods for General and Molecular Microbiology”, American Society for Microbiology, 1994).

In certain embodiments of the invention, the screening assay is performed in a high throughput format, e.g., using microwell plates (e.g., 96-well, 384-well, 1596-well, etc). Such assay plates are commercially available, for example, from Stratagene Corp. (La Jolla, Calif.) and Corning Inc. (Acton, Mass.). High throughput assays may use robotics for various steps such as liquid handling, compound dispensing, plate manipulation, etc. According to these approaches, cells or populations of cells, are dispersed into individual vessels, e.g., wells in a multi-well plate. The number of cells to be added to each well will depend on the size of the wells (i.e., the number of wells per plate) as well as on the method used of the analysis of the screen. Plate readers can be used to detect signals such as optical density, colorimetric or fluorescent readouts, etc.

In certain embodiments of the present invention, the cells used in the inventive screening assays are bacterial cells. Bacteria suitable for use in the practice of the present invention include, but are not limited to, members of the following genuses: Actinomyces, Staphylococcus, Streptococcus, Enterococcus, Erysipelothrix, Neisseria, Branhamella, Listeria, Bacillus, Corynbacterium, Erysipelothrix, Gardnerella, Mycobacterium, Nocardia, Enterobacteriaceae, Escherichia, Salmonella, Shigella, Yersinia, Enterobacter, Klebsiella, Citrobacter, Serratia, Providencia, Proteus, Morganella, Edwardsiella, Erwinia, Vibrio, Aeromonas, Helicobacter, Campylobacter, Eikenella, Pasteurella, Pseudomonas, Burkholderia, Stenotrophomonas, Acinetobacter, Ralstonia, Alcaligenes, Moraxella, Mycoplasma, Legionella, Francisella, Brucella, Haemophilus, Bordetella, Clostridium, Bacteroides, Porphyromonas, Prevotella, Fusobacterium, Borrelia, Chlamydia, Rickettsia, Ehrlichia, Bartonella, Trichomonas, and Treponema.

In certain embodiments of the invention, the bacteria are causative of disease in humans and/or animals. Examples include, but are not limited to, Aeromonas hydrophila, Bacillus subtilis, Escherichia coli, Enterobacter cloacae, Campylobacter jejuni, Haemophilus influenzae, Klebsiella pneumoniae, Klebsiella oxytoca, Legionella pneumophila, Pasteurella multocida, Proteus mirabilis, Proteus vulgaris, Morganella morganii, Helicobacter pylori, Neisseria gonorrhoeae, Pseudomonas aeruginosa, Salmonella enterica, Salmonella typhimurium, Staphylococcus aureus, Staphylococcus epidermidis, Streptococcus pneumoniae, Streptococcus pyogenes, and Streptococcus agalactiae.

In certain embodiments of the invention, the cells used in the inventive screening assays are genetically engineered.

For example, different strains may be used in which a different gene is altered in each strain. Typically, in such embodiments, the strains will be members of a bacterial species (e.g., E. coli or S. aureus) and will be genetically identical except for the genetic alteration. The alteration may, for example, involve deletion of all or part of the gene, so that either (i) no functional gene product is synthesized; (ii) the amount of functional gene product is substantially reduced; or (iii) the gene product has substantially reduced or no activity. The availability of complete genome sequences for a variety of different bacteria has facilitated the development of such strain collections. Deletion or functional inactivation can be achieved using a variety of different methods known in the art. Alternatively or additionally, different stains can be used in which a different gene is over-expressed in each strain. Over-expression can be achieved by a variety of methods including, by introducing an expression vector containing the relevant gene (or the coding portion thereof) into the cells, by using a strong promoter functional in bacterial cells, by integrating a recombinant nucleic acid construct encoding the gene into the bacterial chromosome, or by introducing a gene derived from one bacterial species into a different bacterial species. Methods for generating strains suitable for use in the screening methods of the present invention are known in the art (see, for example, Sambrook et al., Molecular Cloning: A Laboratory Manual, 3^{rd} Ed., Cold Spring Harbor Laboratory Press, Cold Spring Harbor, 2001; Datsenko et al., Proc. Natl. Acad. Sci. USA, 2000, 97: 6640-6645; Murphy, J. Bacteriol., 1998, 180: 2063-2071; Link et al., J. Bacteriol., 1997, 179: 6228-6237; Arigoni et al., Nat. Biotechnol., 1998, 16: 851-856; Lee et al., Appl. Environ. Microbiol., 1999, 65: 1883-1890; Lee et al., Appl. Environ Microbiol., 1998, 64: 4796-4802; Link et al., J. Bacteriol., 1997, 179: 6228-6237; Metcalf et al., Plasmid, 1996, 35: 1-13).

In other embodiments, cells used in the screening methods of the present invention contain one or more mutations that confer resistance to the antibacterial agent used in the screen. For example, the cells may be bacteria having one or more mutations that confer quinolone resistance.

### B—Antibiotic Agents

Screening methods of the invention may be performed to identify compounds that potentiate the activity of members of any class of antibiotic agents. The antibiotic agent can be a broad spectrum or narrow spectrum antibiotic agent. Broad-spectrum antibiotics are antibiotics with activity against a wide range of disease-causing bacteria. Narrow-spectrum antibiotics are effective against only specific families of bacteria. The antibiotic agent may be active against Gram-positive bacteria, Gram-negative bacteria, or both. Gram-positive bacteria are classified as bacteria that retain a crystal violet dye during the grain stain process. Gram-positive bacteria appear blue or violet under a microscope, whereas Gram-negative bacteria appear red or pink. The difference in classification is largely based on a difference in the bacteria's cell wall structure. Alternatively, the antibiotic agent may be active against acid fast bacilli. Acid fast organisms are bacteria that exhibit resistance to decolorization by acids during certain staining procedures involving an acidic alcohol solution (e.g., Ziehl-Neelsen stain).

An antibiotic agent suitable for use in the screening methods of the present invention can exert its antibiotic effect by any of a variety of mechanisms of action (Goodman and Gilman's The Pharmacological Basis of Therapeutics, 10^{th} Ed., McGraw Hill, 2001; Basic and Clinical Pharmacology, B. Katzung (Ed.), McGraw Hill/Appleton & Lange, 8^{th} Ed. (Sep. 21, 2000); Merck Manual of Diagnosis and Therapy, 17^{th} Ed; Physician's Desk Reference, etc., each of which is incorporated herein by reference). For example, an antibiotic agent can act by inhibiting bacterial cell wall synthesis; can act by disrupting cell membrane function; can affect cellular mechanisms of information transfer and protein synthesis; can affect the replication of genetic material by inhibiting nucleic acid synthesis; can interfere with intermediary metabolism; or can exert its antibiotic effect by any combinations of these or other mechanisms of action.

For example, antibiotics suitable for use in the screening methods of the present invention may be selected from the group consisting of bacitracin; the cephalosporins (such as cefadroxil, cefazolin, cephalexin, cephalothin, cephapirin, cephradine, cefaclor, cefamandole, cefonicid, ceforanide, cefoxitin, cefuroxime, cefoperazone, cefotaxime, cefotetan, ceftazidime, ceftizoxime, ceftriaxone, and meropenem); cycloserine; fosfomycin, the penicillins (such as amdinocillin, ampicillin, amoxicillin, azlocillin, bacamipicillin, benzathine penicillin G, carbenicillin, cloxacillin, cyclacillin, dicloxacillin, methicillin, mezlocillin, nafcillin, oxacillin, penicillin G, penicillin V, piperacillin, and ticarcillin); ristocetin; vancomycin; colistin; novobiocin; the polymyxins (such as colistin, colistimathate, and polymyxin B); the aminoglycosides (such as amikacin, gentamicin, kanamycin, neomycin, netilmicin, paromomycin, spectinomycin, streptomycin, and tobramycin), the tetracyclines (such as demeclocycline, doxycycline, methacycline, minocycline, and oxytetracycline); carbapenems (such as imipenem); monobactams (such as aztreonam); chloramphenicol; clindamycin; cycloheximide; fucidin; lincomycin; puromycin; rifampicin; other streptomycins; the macrolides (such as erythromycin and oleandomycin); the fluoroquinolones; actinomycin; ethambutol; 5-fluorocytosine; griseofulvin; rifamycins; the sulfonamides (such as sulfacytine, sulfadiazine, sulfisoxazole, sulfamethoxazole, sulfamethizole, and sulfapyridine); and trimethoprim.

In certain embodiments, the antibiotic agent has activity as a DNA gyrase inhibitor, a topoisomerase inhibitor (e.g., topoisomerase IV inhibitor), or both. Preferably, the DNA gyrase or topoisomerase is a DNA gyrase or topoisomerase that is found in a bacteria, fungus, protozoa, or other parasite. In some embodiments, the antibiotic agent is a quinolone antibiotic.

Quinolone antibiotics are compounds that contain a quinolone or a naphthyridine nucleus with any of a variety of different chains and substituents. Numerous modifications of the originally identified core structures have been made resulting in a large number of compounds with activity against differing groups of bacteria. Quinolone antibiotics are described, e.g., in “Fluoroquinolone Antibiotics”, A. R. Ronald and D. E. Low (Eds.), 2003, Birkhhauser Verlag Basel; A. D. DaSilva et al., Curr. Med. Chem., 2003, 10: 21-39; F. Van Bambeke et al., Clin. Microbiol. Infect., 2005, 11: 156-280; and U.S. Pat. Nos. 3,669,965; 4,563,459; 4,620,007; 4,382,892; 4,985,557 5,053,407; and 5,142,046.

Quinolones have been reported to act by forming a ternary complex with the topoisomerase enzymes and DNA. The lethal effect may be due to enhancement of DNA cleavage and/or blocking of DNA religation following cleavage by the topoisomerase rather than primarily to inhibition of DNA replication. Quinolones increase the intracellular concentrations of the cleavage complexes that are intermediates in the topoisomerase-mediated reactions. The accumulation of permanent double-stranded DNA breaks eventually leads to bacterial death. Resistance to quinolones arises primarily due to a variety of mutations which make the enzymes less sensitive to quinolones or which affect microbial efflux pumps and decrease cellular accumulation of the drug. Quinolone resistance can arise in a step-wise fashion as bacteria accumulate multiple mutations in either the same or different type II topoisomerase subunits.

Quinolone antibiotics include, but are not limited to, any of the antibiotic agents disclosed in the foregoing references including, but not limited to ciprofloxacin, oxolinic acid, cinoxacin, flumequine, miloxacin, rosoxacin, pipemidic acid, norfloxacin, enoxacin, moxifloxacin, gatifloxacin, ofloxacin, lomefloxacin, temafloxacin, fleroxacin, pefloxacin, amifloxacin, sparfloxacin, levofloxacin, clinafloxacin, nalidixic acid, enoxacin, grepafloxacin, levofloxacin, lomefloxacin norfloxacin, ofloxacin, trovafloxacin, olamufloxacin, cadrofloxacin, alatrofloxacin, gatifloxacin, rufloxacin, irloxacin, prulifloxacin, pazufloxacin, gemifloxacin, sitafloxacin, tosulfloxacin, amifloxacin, nitrosoxacin-A, DX-619, and ABT-492. Quinolone antibiotics include fluoroquinolones (e.g., having a fluorine substituent at the C-6 position), and non-fluoroquinolones. Also included within the scope of quinolone antibiotics are derivatives in which a quinolone is conjugated with, e.g., covalently bound to, another core structure. For example, U.S. Pat. No. 6,869,965 discloses compounds in which an oxazolidinone, isoxazolinone, or isoxazoline is covalently bonded to a quinolone.

Included within the scope of quinolone antibiotics that can be used in the screening methods are compounds that have a core structure related to the 4-oxo-1,4-dihydroquinoline and 4-oxo-1,4 dihydronapthyridine systems, e.g., 2-pyridones, 2-naphthyridinones, and benzo[b]napthyridones. 2-pyridones are potent inhibitors of bacterial type II topoisomerases (A. Y. C. Saiki et al., Antimicrob. Agents Chemother., 1999, 43: 1574-1577).

In certain embodiments, the antibiotic agent is a fluoroquinolone. Fluoroquinolones are major drugs in the arsenal to fight infections as they have broad spectrum of activity against Gram-positive and Gram-negative species.

In certain embodiments of the invention, the antibiotic agent is the fluoroquinolone, norfloxacin. In other embodiments of the invention, the antibiotic agent is a fluoroquinolone selected from the group consisting of ciprofloxacin, oxofloxacin and levofloxacin.

In addition to the quinolone antibiotics, a variety of agents are know in the art that inhibit one or more bacterial type II topoisomerase inhibitors, some of which are structurally related to quinolones. Exemplary inhibitors include the coumarins, novobiocin and coumermycin A1, cyclothialidine, cinodine, and clerocidin. Additional compounds that are reported to bind to and/or inhibit gyrase, topoisomerase IV, or both, are disclosed in U.S. Pat. Nos. 6,608,087 and 6,632,809 and in U.S. Pub. Nos. 2004-0043989 and 2005-0054697.

In other embodiments, the antibiotic agent is an aminoglycoside. Aminoglycosides display bacterial, concentration-dependent killing action and are active against a wide range of aerobic Gram-negative bacilli. They are also active against staphylococci and certain mycobacteria. Aminoglycosides work by binding to the bacterial 30S ribosomal subunit, inhibiting the formation of initiation complex and also causing misreading of t-RNA, leaving the bacterium unable to synthesize proteins vital to its growth. The antibiotic agent may be an aminoglycoside, for example, selected from the group consisting of amikacin, gentamicin, kanamycin, neomycin, netilmicin, paromomycin, streptomycin, tobramycin and apramycin.

In certain screening methods of the invention, exposing cells to an antibiotic agent, contacting cells with an antibiotic agent, or incubating cells with an antibiotic agent comprises adding the antibiotic agent to a container (e.g., well of a micro-well plate) containing cells and incubating the cells in the presence of the antibiotic in a suitable culture medium under conditions (e.g., antibiotic concentration, temperature, humidity, etc.) and for a period of time such that the intended role of the antibiotic agent is or can be achieved. More specifically, exposing cells to an antibiotic agent should be carried out under conditions that allow the antibiotic agent to exert its effect(s). Such conditions and period of time are either well known in the art or may readily be determined, for example, empirically, by one of ordinary skill in the art.

The antibiotic may be present at a sub-lethal concentration, at a cytotoxic concentration, at a concentration lower than a cytotoxic concentration, at a concentration lower than a cytostatic concentration, at a concentration that results in a transcriptional response similar to that achieved by a lethal concentration, at a concentration that results in a transcriptional response similar to that achieved by a cytostatic concentration, etc.

The particular antibiotic concentration selected will depend on a variety of parameters including the bacterial species or strains used, whether growth or survival (viability following a period of exposure to the antibiotic) is to be assessed, etc. Typically, the concentrations will be sub-lethal for a growth assay. The concentration may be one that does not significantly reduce bacterial growth but is sufficient to cause at least some alterations in bacterial physiology. For example, the concentration of the antibiotic agent may be one that causes detectable alterations in expression of one or more genes. In certain embodiments of the invention, the concentration selected for a screen employing a growth assay is between about 1% and 5% of the MIC, between about 5% and 10% of the MIC, between about 10% and 25% of the MIC, between about 25% and 50% of the MIC, between about 50% and 75% of the MIC, between about 75% and 95% of the MIC, or any specific sub-range or value within a foregoing range. In certain embodiments of the invention, the concentration of antibiotic agent selected for a screen employing a growth assay reduces growth of wild type bacteria (not having functional inactivation of a gene) to between about 5% and 10%, between about 10% and 25%, between about 25% and 50%, between about 50% and 75%, or between about 75% and 95% of the growth in the absence of the antibiotic or any specific sub-range or value within a foregoing range. In certain embodiments of the invention, the concentration of antibiotic agent selected for a screen employing a survival assay is between about 1 and 2 times the MIC, between about 2 and 5 times the MIC, or between about 5 and 10 times the MIC.

It will be appreciated that a screening method according to the present invention involves comparing growth or survival that the cells incubated in the presence of the antibiotic and candidate compound (test cells) and cells incubated in the presence of the antibiotic alone (control cells) would exhibit under substantially equivalent conditions, particularly with respect to the concentration of the antibiotic and the time of exposure. However, substantially equivalent conditions need not actually be employed in performing the method, provided that the growth or survival results for the test and control cells can be correlated with what would be expected to occur under substantially equivalent conditions.

Substantially equivalent conditions of antibiotic exposure with respect to concentration of the antibiotic agent typically means that the concentrations of antibiotic to which the test and control cells are exposed are within a factor of 2-fold of one another, or that the concentrations would be expected to have substantially the same effect on identical cells (for example, two different concentrations that are both much larger than the MIC would be expected to have substantially the same inhibitory effect even if the absolute concentrations varied by more than a factor of 2, and two different concentrations that are both much smaller than the MIC would be expected to have substantially no effect even if the absolute concentrations varied by more than a factor of 2). Preferably, substantially equivalent antibiotic exposure is exposure at concentrations that differ by no more than a factor of 2. The concentrations may be identical to within experimental error, or the higher concentration may be 110% or less, 120% or less, 130% or less, 140% or less, or 150% or less of the lower concentration. Alternatively, the concentrations may differ by 10% or less, 20% or less, 30% or less, 40% or less, or 50% or less of the MIC for the antibiotic. With respect to time during which exposure occurs, substantially equivalent conditions would typically mean that the length of exposure differs by no more than a factor of 2 and may, for example, differ by 10% or less, 20% or less, 30% or less, 40% or less, or 50% or less of the shorter time of exposure or be substantially identical (i.e., identical to within 2% of the shorter time of exposure). Substantially equivalent conditions may also entail use of the same growth medium, temperature, etc., for cells whose growth or survival is to be compared.

### C—Candidate Compounds

As will be appreciated by those of ordinary skill in the art, any kind of compounds can be tested using the inventive screening methods. A candidate compound may be a synthetic (i.e., non-naturally-occurring) or a natural (i.e., naturally-occurring) compound. A candidate compound may be a single molecule or a complex formed by at least two molecules. Sources for candidate compounds include natural product extracts, collections of synthetic compounds, and compound libraries generated by combinatorial chemistry.

Collections and libraries of compounds are well known in the art. Natural product collections are generally derived from microorganisms, animals, plants, or marine organisms; they include polyketides, non-ribosomal peptides, and/or variants (non-naturally-occurring) thereof (see, for example, D. E. Cane et al., Science, 1998, 82: 63-68). Collections of natural compounds in the form of bacterial, fungal, plant and animal extracts are available from, for example, Pan Laboratories (Bothell, Wash.) or MycoSearch (Durham, N.C.).

Libraries of candidate compounds that can be used in the practice of the present invention may be either prepared or purchased from a number of companies. One representative sample if known as DIVERSet™, available from ChemBridge Corporation (San Diego, Calif.). DIVERSet™ contains between 10,000 and 50,000 drug-like, hand-synthesized small molecules. These compounds are pre-selected to form a “universal” library that covers the maximum pharmacophore diversity with the minimum number of compounds and is suitable for either high-throughput or lower throughput screening. For descriptions of additional libraries, see, for example, Tan et al., Am. Chem. Soc., 1998, 120: 8565-8566, C. D. Floyd et al., Prog. Med. Chem., 1999, 36: 91-168. Numerous libraries are commercially available, e.g., from AnalytiCon USA Inc. (Kingwood, Tex.); 3-Dimensional Pharmaceuticals, Inc. (Exton, Pa.); Tripos, Inc. (St Louis, Mass.); Comgenex (Princeton, N.J.), Brandon Associates (Merrimack, N.H.), Microsource (New Milford, Conn.), and Aldrich (Milwaukee, Wis.). Libraries of candidate compounds have also been developed by and are commercially available from large chemical companies, including, for example, Merck, Glaxo Welcome, Bristol-Meyers-Squibb, Novartis, Monsanto/Searle, and Pharmacia UpJohn.

Libraries of compounds are relatively easy to prepare by traditional automated synthesis, PCR, cloning or proprietary synthetic methods (see, for example, S. H. DeWitt et al., Proc. Natl. Acad. Sci. U.S.A. 1993, 90:6909-6913; E. Erb et al., Proc. Natl. Acad. Sci. U.S.A. 1994; 91: 11422-11426; R. N. Zuckermann et al., J. Med. Chem. 1994, 37: 2678-2685; C. Y. Cho et al., Science, 1993, 261: 1303-1305; Carell et al., Angew. Chem. Int. Ed. Engl. 1994, 33: 2059-2060; Carell et al., Angew. Chem. Int. Ed. Engl. 1994, 33: 2061-2063; and M. A. Gallop et al., J. Med. Chem. 1994, 37: 1233-1251; and P. L. Myers, Curr. Opin. Biotechnol. 1997, 8: 701-707). Libraries may be provided in solution or may be attached to a solid support such as a bead.

In certain embodiments of the invention, the compounds to be tested are synthesized to contain a common core structure. The core structure may be one that characterizes a compound shown to display potentiating activity for a particular antibiotic agent (e.g., using a cell-free or cell-based assay) and/or predicted to display activity, for example, based on computational approaches. Once a library of compounds is screened, subsequent libraries may be generated using those chemical building blocks that possess the features shown in the first round of screen to have potentiating activity. Using this approach, subsequent iterations of candidate compounds will possess an increasing number of those structural and functional features required to potentiate the antibiotic activity of interest, until a group of compounds with high potentiating activity and, optionally, one or more additional desirable properties (e.g., cell permeability) can be found. The present invention encompasses these improved candidate compounds. These compounds can then be further tested for their safety and efficacy in therapeutic use.

Useful potentiating compounds may be found in various classes of chemicals, including heterocycles, peptides, saccharides, steroids, and the like. In certain embodiments, the methods of the present invention are used for identifying potentiating compounds that are small molecules. Preferred small organic molecules have a molecular weight of more than 50 and less than about 2,500 Daltons, preferably less than 600-700 Daltons.

Candidate compounds to be tested and screened by the assays of the invention can be compounds previously unknown to have any pharmacological activity, or can be pharmacologic agents already known in the art. For example, candidate compounds can be selected among drugs or derivatives of drugs known in the art to be useful in the treatment of diseases or pathophysiological conditions caused by the particular microorganism under investigation in the screen.

### D—Identification of Potentiating Compounds

According to certain screening methods of the invention, determination of the ability of a candidate compound to potentiate the activity of an antibiotic agent includes comparison of cell growth or survival in test cells and control cells, wherein test cells are incubated in the presence of the antibiotic agent and candidate compound, while control cells are incubated under the same conditions and for the same period of time except for the presence of the candidate compound.

In such methods, a candidate compound is identified as a potentiating compound of the antibiotic agent if the growth or survival of cells in the presence of the antibiotic agent and the candidate compound is less than the growth of the cells in the presence of the antibiotic agent alone.

Growth or survival can be assessed using cells growing in liquid media or on solid or semi-solid media. Any method known in the art can be used to determine whether an agent (e.g., the antibiotic agent) or combination of agent (e.g., candidate compound and antibiotic agent) inhibits growth, proliferation and/or survival. Examples include measuring optical density in liquid culture, measuring colony formation, or measuring bacterial viability. Bacterial viability can be assessed based on metabolic characteristics such as oxidation/reduction state, ability to metabolize particular substrate(s) or produce particular metabolite(s), or based on membrane integrity, which can be detected by evaluating ability of a bacterial cell to exclude a particular substance such as a detectable molecule (e.g., a fluorescent or luminescent molecule) from the cell interior.

In one embodiment, a commercially available assay such as the LIVE/DEAD BacLight Bacterial Viability assay (Molecular Probes, Invitrogen, Carlsbad, Calif.) is used. This assay utilizes mixtures of SYTO® 9 green fluorescent nucleic acid stain and the red fluorescent nucleic acid stain, propidium iodide. These stains differ both in their spectral characteristics and in their ability to penetrate healthy bacterial cells. When used alone, the SYTO 9 stain labels bacteria with damaged membranes. Propidium iodide, however, penetrates only bacteria with damaged membranes, competing with the SYTO 9 stain for nucleic acid binding sites when both dyes are present. When mixed in recommended proportions, SYTO 9 stain and propidium iodide produce green fluorescent staining of bacteria with intact cell membranes and red fluorescent staining in bacteria with damaged membranes. The background remains virtually non-fluorescent. The ratio of green to red fluorescence intensities therefore provides a quantitative index of bacterial viability. A fluorimeter can be used to detect the fluorescence intensities.

Another suitable assay for determining the number of viable bacterial cells in culture is based on quantitation of the ATP present (ATP is an indicator of metabolically active cells). The BacTiter-Glo™ assay (Promega, Madison, Wis.) is a commercially available assay based on a principle that involves adding a single reagent (BacTiter-GLO™ Reagent) directly to bacterial cells in medium and measuring luminescence.

Many additional assays suitable for assessing bacterial viability are described in “Handbook of Fluorescent Probes and Research Products”, Molecular Probes”, 9^{th} Edition, 2002 and “The Handbook—A Guide to Fluorescent Probes and Labeling Technologies”, Invitrogen, 10^{th} Edition (available at the Invitrogen web site) and can be used in the practice of the screening methods of the present invention.

Reproducibility of the results obtained may be tested by repeating the experiment or, in the case of a high-throughput assay, by incubating cells in more than one well of the assay plate (for example, in triplicate) with the same concentration of the same candidate compound. Additionally, since candidate compounds may be effective at varying concentrations depending on the nature of the compounds and the nature of its mechanism(s) of action, varying concentrations of the candidate compound may be added to different wells containing cells. Generally, concentrations from about 1 fM to about 10 mM are used for screening. Preferred screening concentrations are generally between about 10 μM and about 100 μM.

In certain embodiments, the methods of the invention further involve the use of one or more negative or positive control compounds. A positive control compound may be any molecule, agent, moiety or drug that is known to potentiate the activity of the antibiotic under investigation in the screening method. A negative control compound may be any molecule, agent, moiety or drug that is known to have no significant potentiating effect on the activity of the antibiotic agent. In these embodiments, the inventive methods further comprise comparing the potentiating effects of the candidate compound to the potentiating effect (or lack thereof) of the positive (or negative) control compound. Such negative and positive control compounds may have been identified by methods described herein.

### E—Characterization of Candidate Compounds

As will be appreciated by those skilled in the art, it is generally desirable to further characterize potentiating compounds identified by the inventive screening methods.

For example, if a candidate compound has been identified as a potentiating compound in a given cell culture system (e.g., a particular strain of a particular bacterial species), it may be desirable to test this ability in a different cell culture system (e.g., a different strain of the same bacterial species or in a different bacterial species). Alternatively or additionally, it may be desirable to evaluate the specificity of the candidate compound by testing its ability to potentiate the activity of other members of the same class of antibiotics. It may also be desirable to perform pharmacokinetics and toxicology studies in microbial cells as well as in other cell types (e.g., mammalian cells).

Candidate compounds identified by screening methods of the invention may also be further tested in assays that allow for the determination of the compounds' properties in vivo. Suitable animal models include animal models of bacterial infection.

Examples of animal models include, but are not limited to, animal models for Helicobacter pylori infection (A. Lee, Mol. Med. Today, 1999, 5: 500-501); pneumococcal pneumonia (E. Nuermberger, Pharmacol., 2005, 25: 134S-139S); pulmonary infection (I. A. Bakker-Woudenberg, J. Microbiol. Methods, 2003, 54: 295-313); S. typhimurium-induced enterocolitis (S. Hapfelmeier and W. D. Hardt, Trends Microbiol., 2005, 13: 497-503); Staphylococcus aureus-induced pathogenesis (E. Brouillette and Malouin, Microbes Infect., 2005, 7: 560-568; J. Garcia Lara et al., FEMS Immunol. Med. Microbiol., 2005, 43: 311-323); Chlamydia trachomatis infections in the female genital tract (D. Vanrompay et al., Drugs Today, 2006, 42 (Suppl. A): 55-63); Salmonella infections (P. Matroeni and M. Sheppard, Microbes Infect., 2004, 6: 398-405; R. L. Santos et al., Microbes Infect., 2001, 3: 1335-1344); bacterial meningitis (R. Paul et al., Arch. Immunol. Ther. Exp., 203, 52: 315-326); tuberculosis (J. L. Flynn et al., Tuberculosis, 2003, 83: 116-118; I. M. Orme, Tuberculosis, 2003, 83: 112-115); Chlamydia pneumonia-induced atherosclerosis (I. W. Fong, J. Infect. Dis., 2000, 181: S515-S518; I. A. Campbell et al., J. Infect. Dis., 2000, 181: S508-S513; I. A. Campbell and C. Kuo, Am. Heart J., 1999, 138: S516-S518); infection-mediated vasculitis (A. J. Dal Canto et al., Curr. Opin. Rheumatol., 1999, 11: 17-23) and Porphyromonas gingivalis-mediated periodondal disease (C. A. Genco et al., Trends Microbiol., 1998, 6: 444-449).

### II. Potentiating Compounds

The present invention provides compounds that potentiate the activity of antibiotic agents. An inventive potentiating compound can potentiate the activity of a single antibiotic agent or of more than one antibiotic agent (e.g., of several non-related antibiotic agents, or several structurally-related antibiotic agents, and/or several mechanistically-related antibiotic agents). Preferably, the potentiating compounds of the present invention are identified using an inventive screening method.

In particular, the present invention provides potentiating compounds that have been identified by a screening method using norfloxacin, a member of the fluoroquinolone family (as described in Example 1). More specifically, the Applicants have generated dose response growth curves to identify a suitable concentration of norfloxacin to use in a screen to identify compounds that potentiate its antibacterial activity. The chosen concentration (50 ng/mL) was sub-lethal in the sense that it allows the cells to grow in the presence of quinolone but still is sufficient to induce a response at the transcriptional level that is characteristic of quinolones, as measured by Affymetrix micro-array technology. Without wishing to be bound by any theory, this concentration of norfloxacin may affect molecular targets (i.e., bacterial genes and their mRNA and/or protein expression products) that are important for the activity of the compound.

The cell-based screening assay (E. coli, Strain MG1655 K12—see T. Baba et al., “Construction of Escherichia coli K-12 in-frame, single-gene knockout mutants: the Keio collection”, Mol. Syst. Biol., 2006, 2:2006.0008. Epub 2006 Feb. 21, which is incorporated herein by reference in its entirety) was performed with over 1,200 compounds alone and in combination with norfloxacin. These 1,200 compounds belong to the CMLD library developed by Boston University. The assay compared cell growth in norfloxacin alone to cell growth in the presence of the same concentration of norfloxacin and the candidate compound. Lack of growth, or poor growth, in the presence of norfloxacin and a candidate compound, relative to growth in the presence of norfloxacin alone, indicated a positive result, i.e., the candidate compound was identified as a likely potentiating compound of norfloxacin activity (see Example 1).

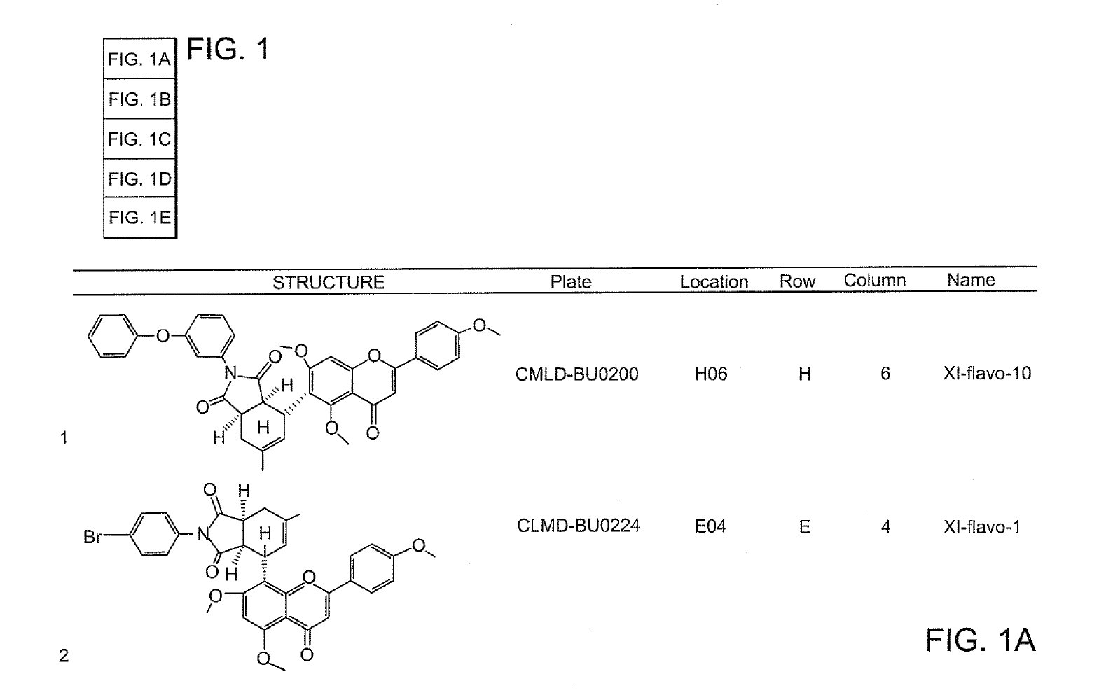

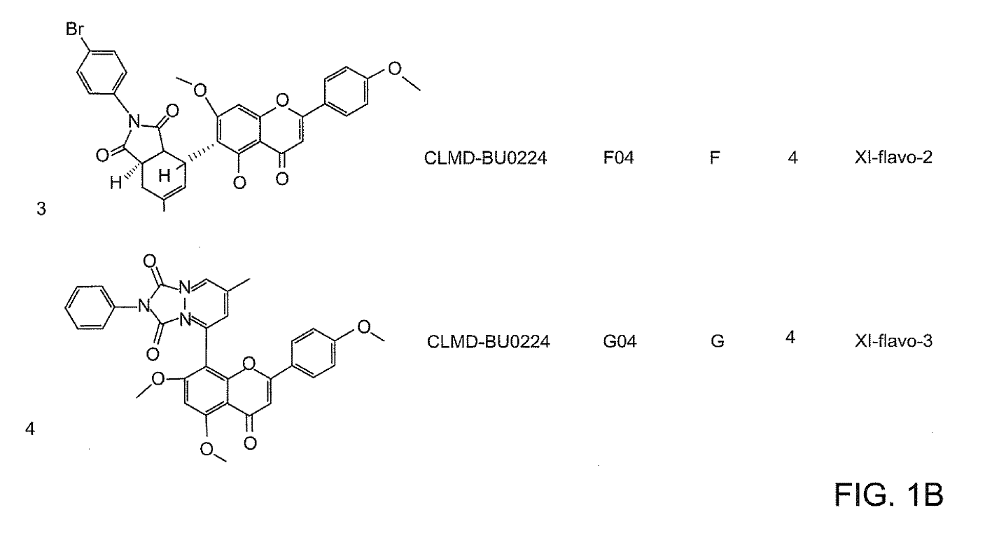

The screen identified a compound that significantly potentiates norfloxacin activity. The compound is presented as compound 8 in FIG. 1 and is also shown in FIG. 2. It was designated CB101. CB101 has a flavone core structure. Flavones are a class of compounds that are known to display ATP antagonist activity. CB101 does not itself appear to affect growth of E. coli, yeast, or mammalian cells over a range of concentrations suggesting that this class of compounds is suitable for use in a therapeutic context. The Applicants have subsequently found that the enantiomers (the + and − forms) of CB101 exhibit similar activity in potentiating the activity of ciprofloxacin (see Example 3).

The screen further identified compounds related in structure to CB101, some of which displayed higher potentiating activity (i.e., higher growth inhibition than CB101), demonstrating the clear potential to develop derivatives of compounds identified in an initial screen. The results also suggested that compounds of other structural classes that are ATP antagonists may potentiate quinolone activity.

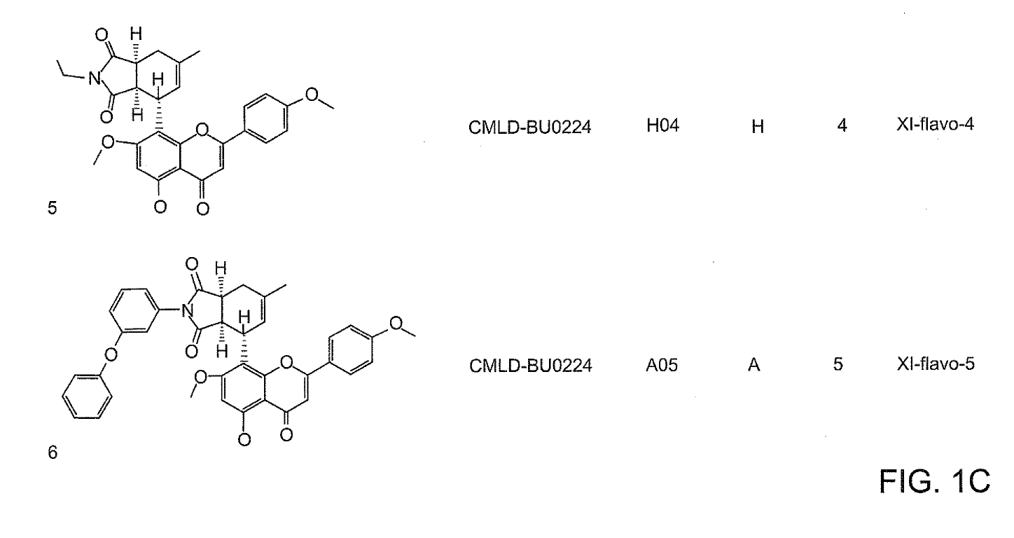

The screen further identified a compound designated as BU332_G10, which has a terpene core structure, as a potentiator of norfloxacin activity (see FIG. 3). Thus, the first round of screening identified two classes of compounds that potentiate quinolone activity.

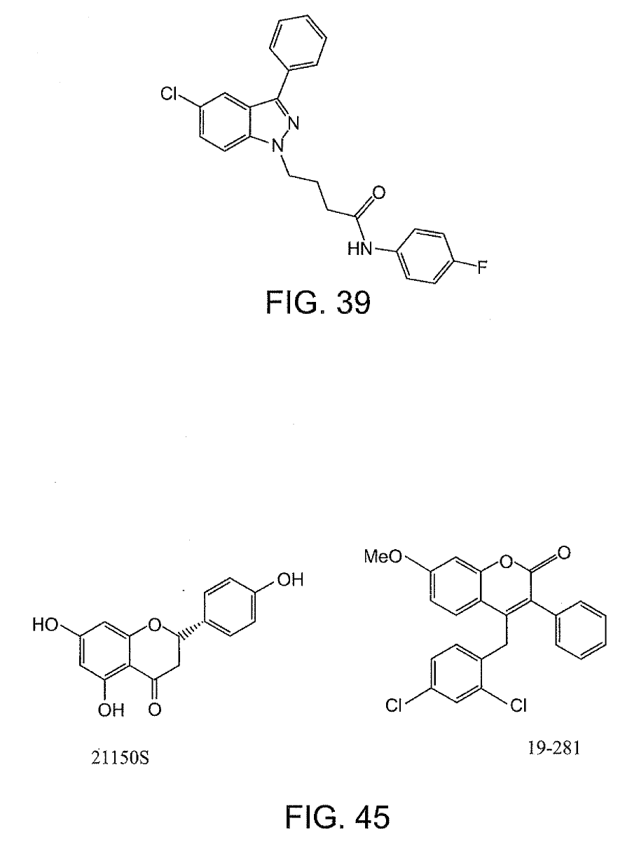

Based on these results, the Applicants have suggested about 200 compounds for screening. These suggestions were mostly based on structural considerations. Several of these compounds that were commercially available were selected for screening as they were representative of the various different structural motifs that have been reported as kinase antagonists. The selected compounds included 20 flavones, 19 isoflavones, 9 coumarins, and 48 heterocyclic compounds (as show on FIG. 38).

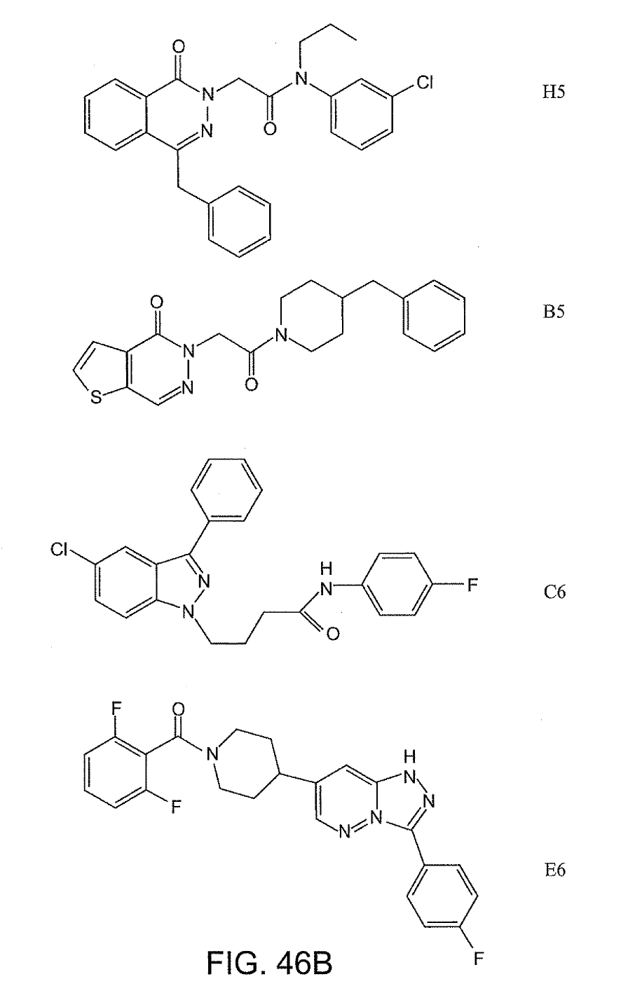

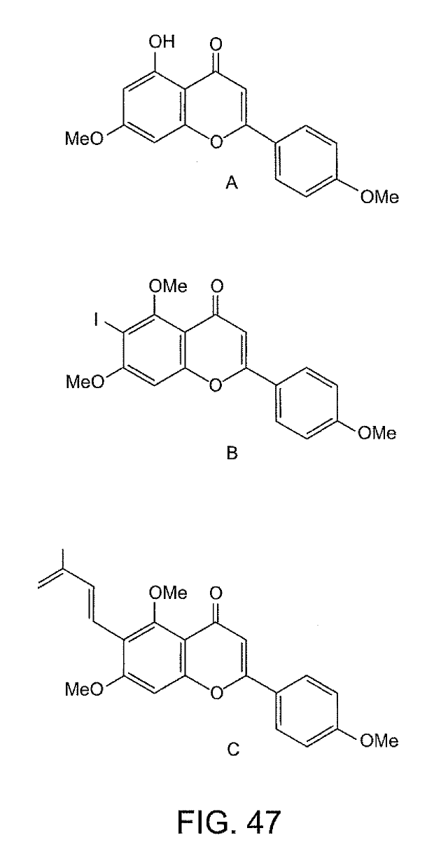

The selected compounds were screened for quinolonoe potentiation. While none of the isoflavones were found to be active, one flavone (the structure of which is presented on FIG. 45), one coumarin (the structure of which is presented on FIG. 45), and eight heterocyclic derivatives (see FIGS. 46(A) and (B)) were found to exhibit significant activity.

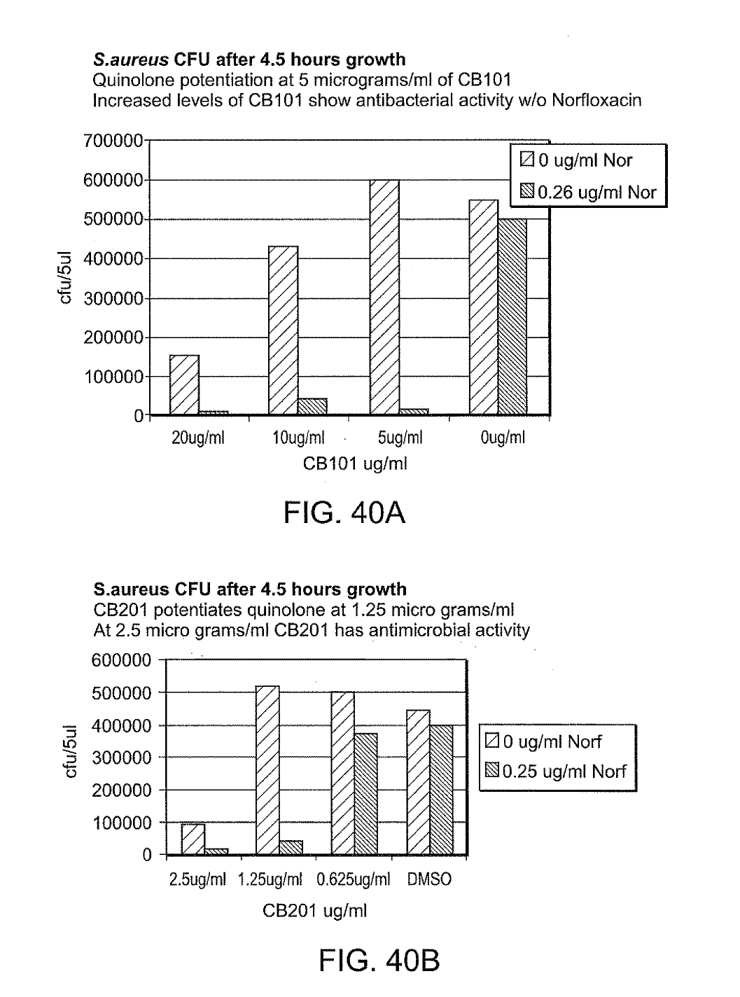

In particular, the screen of this “suggested library” identified compound CB201, the structure of which is presented on FIG. 39 (see Example 4).

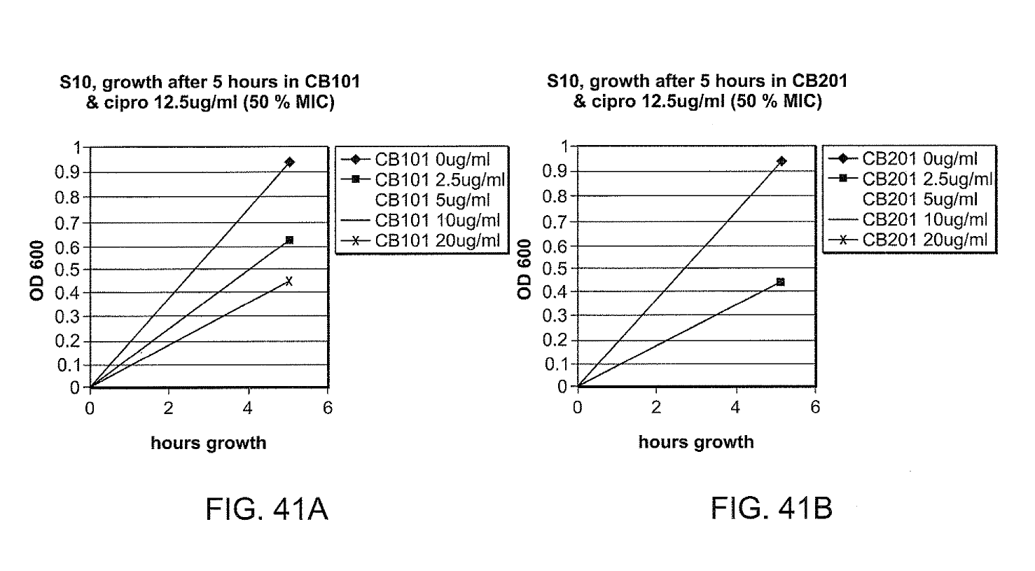

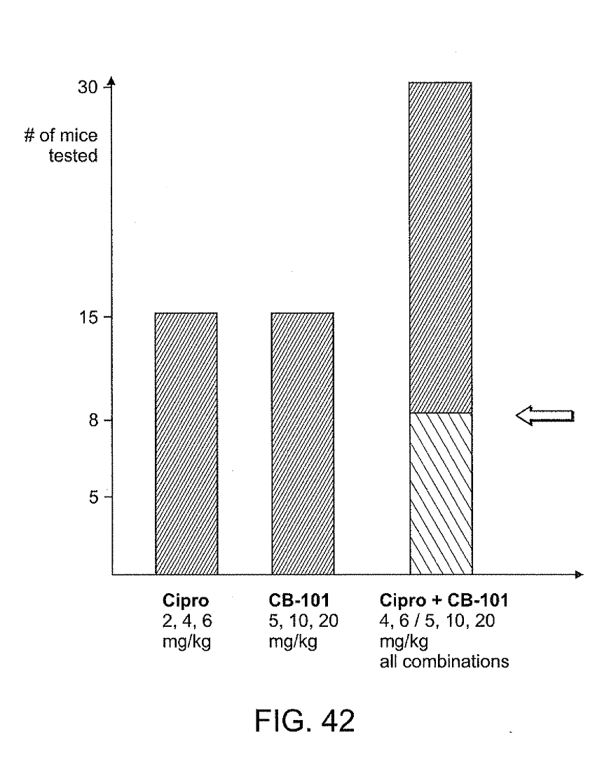

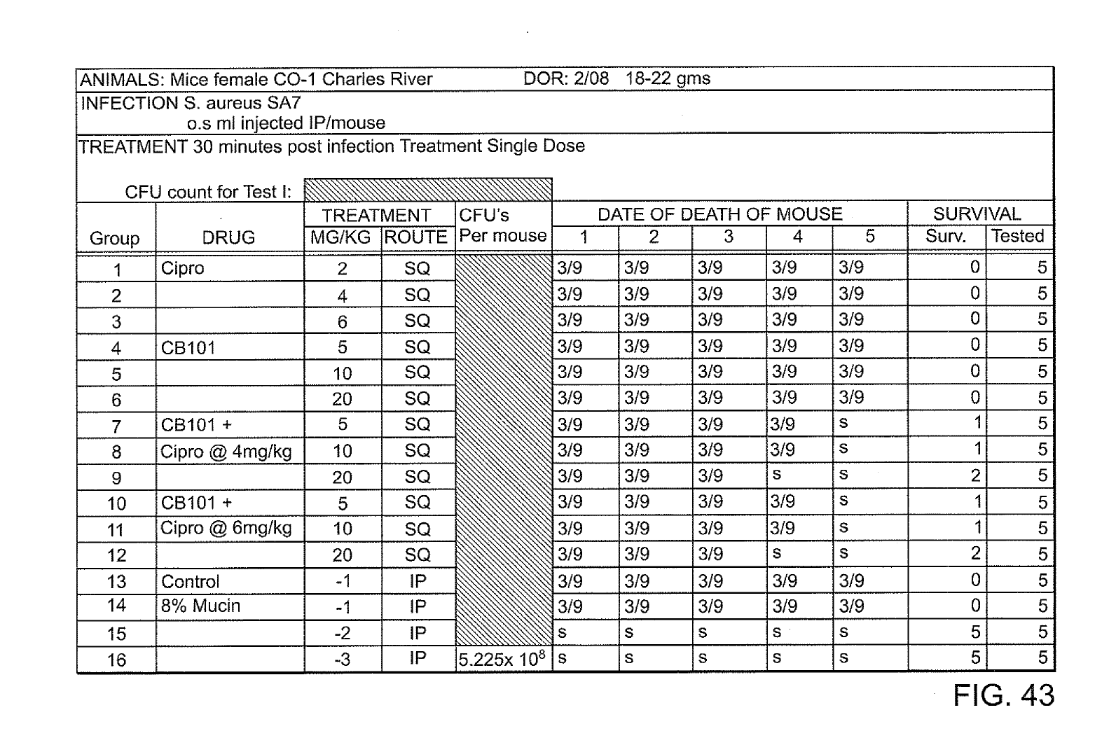

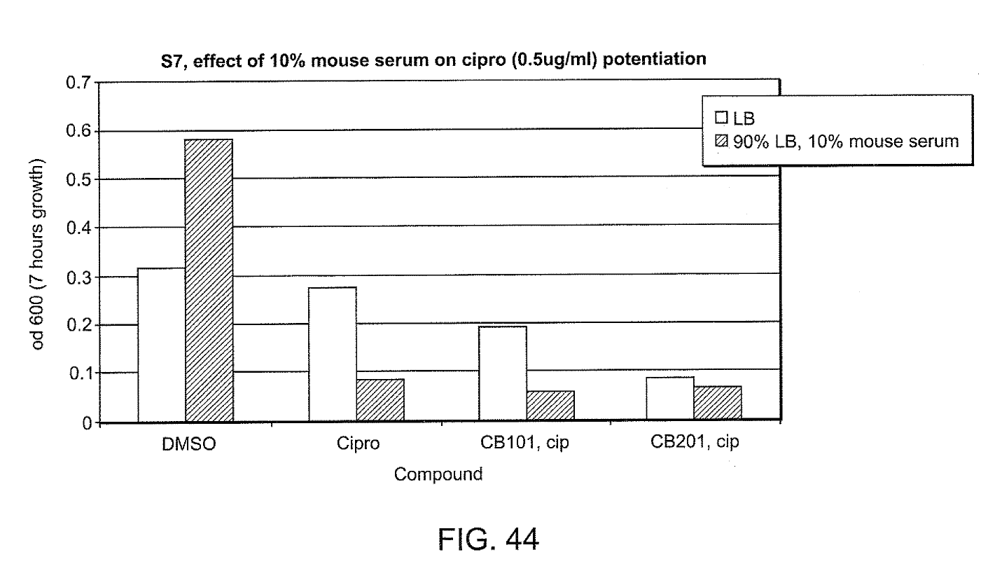

CB101 and CB201 were found to potentiate Norfloxacin in S. aureus (see Example 5). More specifically, both compounds were observed to potentiate the quinolone at low concentration and to exhibit anti-growth activity at higher concentration in S. aureus (see FIG. 40). Both CB101 and CB201 were found to work at low concentrations (in the lower microgram/mL range) against Staphylococcus clinical isolates resistant to quinolones (see Example 5 and FIG. 41). Animal tests have confirmed the potentiating activity of these compounds. In particular, CB101 was shown to potentiate the activity of ciprofloxacin after S. aureus infection of mice with moderately fluoroquinolone resistant S7 Staphylococcus isolate (see Example 6 and FIG. 42 and FIG. 43).

The present invention provides CB101 and CB201 as quinolone potentiating compounds and also encompasses any active (i.e., exhibiting potentiating activity) derivatives, analogs and prodrugs thereof as well any active synthetic intermediates and active derivatives thereof.

### III. Pharmaceutical Compositions and Delivery Vehicles and Methods

Suitable preparations, e.g., substantially pure preparations of the compounds may be combined with pharmaceutically acceptable carriers, diluents, solvents, etc., to produce an appropriate pharmaceutical composition. Accordingly, the present invention provides a variety of pharmaceutical compositions for administration to a subject comprising (i) an antibiotic potentiating compound; and (ii) a physiologically acceptable carrier, adjuvant or vehicle. The present invention also provides pharmaceutical compositions suitable for administration to a subject comprising (i) an antibiotic potentiating compound; (ii) an antibiotic agent whose activity is potentiated by the compound; and (iii) a physiologically acceptable carrier, adjuvant or vehicle.

It is to be understood that the pharmaceutical compositions of the invention, when administered to a subject, are preferably administered for a time and in an amount sufficient to treat the disease or condition for whose treatment they are administered (e.g., a viral, bacterial, fungal, protozoal, or other parasitic infection).

Further provided are pharmaceutical compositions comprising a pharmaceutically acceptable derivative (e.g., a prodrug) of any of the compounds of the invention, by which is meant any non-toxic salt, ester, salt of an ester or other derivative of a compound of this invention that, upon administration to a recipient, is capable of providing, either directly or indirectly, a compound of this invention or an active metabolite or residue thereof. As used herein, the term “active metabolite or residue thereof” refers to a metabolite or residue that is also able to potentiate the activity of an antibiotic agent.

In various embodiments of the invention an effective amount of the pharmaceutical composition is administered to a subject by any suitable route of administration including, but not limited to, intravenous, intramuscular, by inhalation, by catheter, intraocularly, orally, rectally, intradermally, by application to the skin, etc. Thus, the inventive compositions may be formulated for delivery by any available route including, but not limited to, parenteral, oral, by inhalation to the lungs, nasal, bronchial, ophthalmic, transdermal (topical), transmucosal, rectal, and vaginal routes. The term “parenteral”, as used herein, includes subcutaneous, intravenous, intramuscular, intra-articular, intra-synovial, intrasternal, intrathecal, intrahepatic, intrelesional, and intracranial injection or infusion techniques.

Physiologically acceptable carriers, adjuvants or vehicles that may be used in the compositions of the present invention include, but are not limited to, ion exchangers, alumina, alumina stearate, licithin, serum proteins, such as human serum albumin, buffer substances such as phosphates, glycine, sorbic acid, potassium sorbate, partial glyceride mixtures of saturated vegetable fatty, acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulase-based substances, polyethylene glycol, sodium carboxymethylcellulose, polyacrylates, waxes, polyethylene-polyoxypropylene-block polymers, polyethylene glycol and wool fat.

Solvents, dispersion media, coatings, antibacterial and antifungal agents, isotonic and absorption delaying agents, and the like, compatible with pharmaceutical administration may be included. Supplementary active compounds, e.g., compounds independently active against the disease or clinical condition to be treated, or compounds that enhance activity of a compound, can also be incorporated into the compositions.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentaneproprionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroethanoesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g., sodium and potassium), alkaline earth metal (e.g., magnesium), ammonium and N⁺(C1-4 alkyl)₄ salts. This invention also envisions the quaternization of any basic nitrogen-containing groups of the compounds disclosed herein. Water or oil-soluble or dispersible products may be obtained by such quaternization.

A pharmaceutical composition is generally formulated to be compatible with its intended route of administration. Solutions or suspensions used for parenteral (e.g., intravenous), intramuscular, intradermal, or subcutaneous application can include the following compounds: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloride acid or sodium hydroxide. The parenteral preparation can be enclosed in ampoules, disposable syringes or multiple vials made of glass or plastic.

Pharmaceutical compositions suitable for injectable use typically include sterile aqueous solutions (where water soluble) and dispersions and sterile powders for the extemporaneous preparation of sterile injectable solutions or dispersions. For intravenous administration, suitable carriers include physiological saline, bacteriostatic water, Cremophor-EL™ (BASF, Parsipanny, N.J.), phosphate buffered saline (PBS), or Ringer's solution.

Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxythylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In all cases, the compositions should be sterile, if possible, and should be fluid to the extent that easy syringability exists.

Preferred pharmaceutical formulations are stable under the conditions of manufacture and storage and must be preserved against the contaminating action of microorganisms such as bacteria and fungi. In general, the relevant carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols, such as mannitol, sorbital, sodium chloride in the composition. Prolonged absorption of injectable compositions can be brought about by including in the composition an agent which delays absorption, for example, aluminum monostearate and gelatin. Prolonged absorption for oral compositions can be achieved by various means including encapsulation.

Sterile injectable solutions can be prepared by incorporating the active compound in the require amount of an appropriate solvent with one ingredient or a combination of ingredients enumerated herein, as required, followed by filtered sterilization. Preferably, solutions for injection are free of endotoxin. Generally, dispersions are prepared by incorporating the active compound into a sterile vehicle which contains a basic dispersion medium and the required other ingredients from those enumerated herein. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash.

Pharmaceutically compatible binding agents and/or adjuvant materials can be included as part of the composition. Tablets, pills, capsules, troches and the like can contain any of the following ingredients, or compounds of a similar nature: a binder such as microcrystalline cellulose, gum tragacanth or gelatin; an excipient such as starch or lactase, a disintegrating agent such as alginic acid, Primogel, or corn starch; a lubricant such as magnesium stearate or Sterote, a glidant such as a colloidal silicon dioxide; a sweetening agent such as sucrose or saccharin, or a flavoring agent such as peppermint, methyl salicylate, or orange flavoring. Formulations for oral delivery may advantageously incorporate agents to improve stability within the gastrointestinal tract and/or to enhance absorption.

For administration by inhalation, the inventive compositions are preferably delivered in the form of an aerosol spray from a pressured container or dispenser which contains a suitable propellant, e.g., a gas such as carbon dioxide, or a nebulizer. Liquid or dry aerosol (e.g., dry powders, large porous particles, etc.) can be used. The present invention also contemplates delivery of compositions using a nasal spray.

For topical application, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyethylene, polyoxypropylene compound, emulsifying wax and water. Alternatively, the pharmaceutical compositions can be formulated in a suitable lotion or cream containing the active compounds suspended or dissolved in one or more pharmaceutically acceptable carriers. Suitable carriers include, but are not limited to, mineral oil, sorbitan monostearate, polysorbate 60, cetyl esters wax, cetearyl alcohol, 2-cetyldodecanol, benzyl alcohol and water.

For local delivery to the eye, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions of the present invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

For transdermal administration, the active compounds are formulated into ointments, salves, gels or creams, as generally known in the art.

The compounds can also be prepared in the form of suppositories (e.g., with conventional suppository bases such as cocoa butter and other glycerides) or retention enemas for rectal delivery.

In addition to the agents described above, in certain embodiments of the invention, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polyethers, and polylactic acid. Methods for the preparation of such formulations will be apparent to those skilled in the art. Certain of materials can also be obtained commercially from Alza Corporation and Nova Pharmaceuticals, Inc. Liposomal suspensions can also be used a pharmaceutically acceptable carriers. These can be prepared according to methods known to those skilled in the art, for example, as described in U.S. Pat. No. 4,522,811 and other references listed herein. Liposomes, including targeted liposomes (e.g., antibody targeted liposomes) and pegylated liposomes have been described (C. B. Hansen et al., Biochem. Biophys. Acta, 1995, 1239: 133-144; V. P. Torchilin et al., Biochem. Biophys. Acta, 2001, 1511: 397-411; T. Ishida et al., FEBS Lett., 1999, 460: 129-133). One of ordinary skill in the art will appreciate that the materials and methods selected for preparation of a controlled release formulation, implant, etc., should be such as to retain activity of the compound. For example, it may be desirable to avoid excessive heating of polypeptides, which could lead to denaturation and loss of activity.

It is typically advantageous to formulate oral or parenteral compositions in dosage unit form for ease of administration and uniformity of dosage. Dosage unit form as used herein refers to physically discrete units suited as unitary dosages for the subject to be treated; each unit containing a pre-determined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED50. Compounds which exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the methods of the invention, the therapeutically effective dose (e.g., dose that is therapeutically effective to achieve a desired degree of antibiotic potentiation) can be estimated initially from cell culture assays. A dose can be formulated in animal models to achieve a circulating plasma concentration that include the IC₅₀ (e.g., the concentration of the test compound which achieves a half-maximal inhibition of symptoms, half-maximal inhibition of growth or survival of an infectious agent, etc.) as determined in cell culture. Such information can be used to more accurately determine useful doses in humans. Levels in plasma can be measured, for example, by high-performance liquid chromatography (HPLC).

A therapeutically effective amount of a pharmaceutical composition typically ranges from about 0.001 to 100 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The pharmaceutical composition can be administered at various intervals and over different periods of time as required, e.g., multiple times per day, daily, every other day, once a week for between about 1 to 10 weeks, between about 2 to 8 weeks, between about 3 to 7 weeks, about 4, 5, or 6 weeks, etc. The skilled artisan will appreciate that certain factors can influence the dosage and timing required to effectively treat a subject, including, but not limited to, the severity of the disease or disorder, previous treatments, the general health and/or age of the subject, and other diseases present. Generally, treatment of a subject with an inventive composition can include a single treatment or, in many cases, can include a series of treatments. It will be appreciated that a range of different dosage combinations (i.e., doses of the antibiotic and antibiotic potentiating agent) can be used.

Exemplary doses include milligram or microgram amounts of the inventive compounds per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). For local administration (e.g., intranasal), doses much smaller than these may be used. It is furthermore understood that appropriate doses depend upon the potency of the agent, and may optionally be tailored to the particular recipient, for example, through administration of increasing doses until a pre-selected desired response is achieved. It is understood that the specific dose level for any particular subject may depend upon a variety of factors including the activity of the specific compound employed, the age, body weight, general health, gender, and diet of the subject, the time of administration, the rate of excretion, any drug combination, and the degree of expression or activity to be modulated.

The invention further provides pharmaceutical compositions comprising two or more compounds of the invention and, optionally, one or more antibiotic agents. The invention further provides pharmaceutical compositions comprising one or more compounds of the invention, optionally one or more antibiotic agents, and an additional active agent. The additional active agent may be an antibiotic that has a different mechanism of action to that of the antibiotic that is potentiated by the compound.

### IV. Applications

Antibiotic potentiating agents and compositions containing them can be used to inhibit growth of bacteria of a wide variety of types including, but not limited to, members of any bacterial genus or species mentioned herein.

The antibiotic potentiating agents and compositions containing them can be used to inhibit bacterial growth and/or survival in a variety of contexts. For example, they may be employed to inhibit growth and/or survival of bacteria maintained in cell culture or inhabiting locations in the environment, e.g., inert surfaces, clothing, towels, bedding, utensils, etc. Of particular interest are fomites, i.e., inanimate objects that may be contaminated with disease causing microorganisms and may serve to transmit disease to a human or animal. Such locations or objects can be contacted with a solution containing the potentiating compound and an antibiotic that is potentiates. The antibiotic potentiating compounds, antibiotics that they potentiate, and/or compositions containing them can be added to food or water, particularly for the prevention of bacterial disease in animals.

An antibiotic agent and a compound that potentiates the antibiotic (e.g., a quinolone antibiotic and a quinolone potentiating compound) may be administered in combination to a subject in need thereof, e.g., a human or animal suffering from or at risk of a bacterial infection. The antibiotic agent and the antibiotic potentiating compound (e.g., a quinolone antibiotic and a quinolone potentiating compound) may be components of a single pharmaceutical composition or may be administered in individual pharmaceutical compositions. They may be administered using the same route of administration or different routes of administration. In certain embodiments of the present invention, a unit dosage form containing a pre-determined amount of a quinolone antibiotic and a pre-determined amount of a quinolone potentiating compound is administered.

A therapeutic regimen that includes an antibiotic and an antibiotic potentiating compound may (i) allow the use of a reduced daily dose of the antibiotic without significantly reducing efficacy; and/or (ii) allow the use of a shorter course of administration of the antibiotic without significantly reducing efficacy; and/or (iii) be effective against a microorganisms species or strain that would otherwise be resistant to the antibiotic when used at clinically tolerated doses, e.g., conventional doses.

Infections and infection-related conditions that can be treated using an antibiotic potentiating compound and an antibiotic, according to the present invention, include, but are not limited to, pneumonia, meningitis, sepsis, septic shock, sinusitis, otitis media, mastoiditis, conjunctivitis, keratitis, external otitis (e.g., necrotizing otitis externa and perichodritis), laryngeal infections (e.g., acute epiglottitis, croup and tuberculous laryngitis), endocarditis, infections of prosthetic valves, abscesses, peritonitis, infectious diarrheal diseases, bacterial food poisoning, sexually transmitted diseases and related conditions, urinary tract infections, pyelonephritis, infectious arthritis, osteomyelitis, infections of prosthetic joints, skin and soft tissue infections, oral infections, dental infections, nocardiosis and actinomycosis, mastitis, brucellosis, Q fever, anthrax, wound infections, etc.

In certain embodiments of the invention, an antibiotic potentiating compound and an antibiotic that it potentiates are used to treat or prevent infection associated with an indwelling device. Indwelling devices include surgical implants, prosthetic devices, and catheters, i.e., devices that are introduced to the body of an individual and remain in position for an extended period of time. Such devices include, of example, artificial joints, heart valves, pacemakers, defibrillators, vascular grafts, vascular catheters, cerebrospinal fluid shunts, urinary catheters, continuous ambulatory periotoneal dialysis (CAPD) catheters, spinal rods, implantable pumps for medication delivery, etc. Potentiating compounds identified by the methods of the invention can be applied to, coated on, imbedded in, or otherwise combined with an indwelling device to prophylactically prevent infections, optionally together with an antibiotic. Alternatively, a potentiating compound of the invention may be administered to a subject, e.g., by injection to achieve a systemic effect shortly before insertion of an indwelling device. The antibiotic to be potentiated could be applied, coated on, imbedded in, or otherwise combined with an indwelling device or may also be delivered systematically. Of course local delivery of the potentiating compound and/or antibiotic may also be employed. Treatment may be continued after implantation of the device during all or part of the time during which the device remains in the body and, optionally thereafter. Agents of this invention may be used in combination with an antibiotic prophylactically prior to dental treatment or surgery.

Alternatively, a potentiating compound of the present invention and an antibiotic that it potentiates can be used to bathe an indwelling device immediately before insertion and/or to bathe wounds or sites of insertion. Exemplary concentrations useful for these purposes range between 1 μg/mL to 10 μg/mL for bathing of wounds or indwelling devices.

Diagnostic methods for determining whether a subject is suffering from or at risk of suffering from a microbial infection are well known in the art, and any such method can be used to identify a suitable subject for administration of an antibiotic and a compound that potentiates the antibiotic. Methods include diagnostic diagnosis based at least in part on symptoms, imaging studies, immunodiagnostic assays, nucleic acid based diagnostics and/or isolation and culture of potentially causative microorganisms from samples such as blood, urine, sputum, synovial fluid, cerebrospinal fluid, pus, or any sample of body fluid or tissue. The inventive methods can include a step of identifying a subject suffering from or at risk of developing a microbial infection, a step of selecting a therapeutic regimen based at least in part on the identity or suspected identity of the microorganism and/or the location or characteristics of the infection. In certain embodiments of the invention, the method includes determining that the subject has a significant likelihood (e.g., at least 5%) of suffering from or being at risk of infection by a microorganism that is resistant to one or more antibiotics and that antibiotic potentiation is advisable.

A subject is at risk of an infection in any of a variety of circumstances. The term “at risk of” implies at increased risk of, relative to the risk such subject would have in the absence of one or more circumstances, conditions, or attributes of that subject, and/or (in the case of humans) relative to the risk that an average, healthy member of the population would have. Specific examples of conditions that place a subject “at risk” include, but are not limited to, immunodeficiencies (particularly those affecting the humoral or non-specific (innate) immune system), prior treatment with antibiotics that may have reduced or eliminated normal microbial flora, treatment with agents that suppress the immune system (e.g., cancer chemotherapy, immunosuppressive agents), chronic diseases such as diabetes or cystic fibrosis, surgery or other trauma, infancy or old age, occupations or living conditions that entail exposure to pathogenic microorganisms, etc.

While it is anticipated that the antibiotic potentiating compound is identified according to the inventive methods will find particular use for inhibiting the growth and/or survival of microorganisms, they may also be used for other purposes. For example, a compound identified according to the present invention may potentiate a therapeutic agent used in treating a disease other than a microbial infection. Agents that are used to inhibit mammalian topoisomerases are of use for the treatment of a variety of cancers. Exemplary agents include camptothecins (e.g., irinotecan and topotecan) and edotecarin (which inhibit mammalian type I topoisomerase), and ectoposide (a mammalian type II topoisomerase inhibitor). Without wishing to be bound by any theory, compounds that potentiate a microbial topoisomerase inhibitor may also potentiate an agent that inhibits mammalian topoisomerase. Such agents may therefore be of use in cancer chemotherapy regimens that employ a mammalian topoisomerase inhibitor.

### EXAMPLES

The following examples describe some of the preferred modes of making and practicing the present invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

### Example 1

### Compound Screening in E. coli, Strain MG1655 K12 and Identification of Compound CB101

Briefly, the growth assay included comparing samples after overnight growth in presence of norfloxacin (growth) to the same samples at the same plate/well location in the presence of both norfloxacin (50 ng/mL) and test compound (e.g., compound X). The amount of norfloxacin selected for the screen is not sufficient to kill and/or inhibit growth by itself. Each well contained a different test compound but at the same concentration. Lack of poor growth in the well with both norfloxacin and compound X with respect to its counterpart without compound X was called a positive.

Growth inhibition was seen in the presence of both norfloxacin and 224_C05 (═CB101) combined, some derivatives of CB101 and a compound of another class (332-G10). All growths (every well) were quantified using a Tecam spectrophotometer at a wavelength of 600 nm.

### Methods

Compounds were housed in wells in microwell plates in two different amounts: 100 μg (100 μg plate) and 37.5 μg (37.5 μg plate). For the 100 μg plates, compounds were resuspended in 50 μL of DMSO (2 μg/μL). 5 μL were used for each 250 culture. For the 37.5 μg plates, compounds were resuspended in 18 μL of DMSO (2 μg/μL). 4 μL were used for each 200 culture. The final amount of compound was 50 μg/mL for each unknown compound in the screen.

An MG1655 K12 E. Coli culture (T. Baba et al., “Construction of Escherichia coli K-12 in-frame, single-gene knockout mutants: the Keio collection”, Mol. Syst. Biol., 2006, 2:2006.0008. Epub 2006 Feb. 21, which is incorporated herein by reference in its entirety) was grown overnight in LB. A 75 mL day culture was started with a 1/100 dilution of the overnight culture. The day culture was grown at 37° C. with shaking to early log phase (OD(600 nm)˜0.25). 5 μL of each compound was added to a well of 96-well plates, one compound per well. 245 μL of a 1/1000 early log culture (diluted in LB) was added to each well containing 5 μL of compound in the screening plates. Two sets of plates were used per compounds: one with compounds only (control growth plate), the other with norfloxacin and compound (test plate, to evaluate growth or lack of growth).

The starting OD(600 nm) of each plate was measured. Plates were left to grow at 37° C. for 16 hours with no movement. The OD(600 nm) of each plate was again measured. The change in OD(600 nm) was calculated for the compound+norfloxacin vs. the compound only control.

Compound 224_C5 showed a clear inhibition of growth. This result was also observable by visual inspection. Compound at location 224_C5 was renamed CB101.

In addition to identifying CB101, the screen allowed identification of several derivatives with the following growth response compared to the norfloxacin only plate data:

| Compound | Inhibitory Ratio |
|---|---|
| CMLD-BU0224 C05 | 33% |
| CMLD-BU0224 F04 | 66% |
| CMLD-BU0200 H06 | 84% |
| CMLD-BU0224 E04 | 88% |
| CMLD-BU0224 E05 | 93% |
| CMLD-BU0224 D05 | 94% |
| CMLD-BU0224 G04 | 105% |
| CMLD-BU0224 H04 | 108% |
| CMLD-BU0224 A05 | 67% |
| CMLD-BU0224 B05 | 94% |

Another compound of a different class (CMLD-BU332-G10) also caused a reduction in growth with a score of 51% compared to the norfloxacin alone plate.

The combination of both sub-lethal amounts of norfloxacin and CB101 was sufficient to inhibit growth in liquid and in rich medium. Compound CB101 does not appear to increase the killing rate of a sub-lethal amount of norfloxacin. The addition or synergistic effect of CB101 with norfloxacin was not detected with sub-lethal amounts of spectinomycin and kanamycin (data not shown). These last two compounds are protein synthesis inhibitors. The potentiation activity of CB101 appears to be specific to norfloxacin, quinolones, and/or DNA damaging agents (e.g., UV, mitomycin C).

CB101 was tested against other types of cells and does not affect E. coli, yeast and mammalian cell growth by itself, suggesting that this class of compounds is suitable for use in a therapeutic context.

The more precise elucidation of the mode of action of CB101 will likely allow identification of its target(s) and pathway(s). Using genomics, proteomics technologies such as transcription profiling (microarrays), the genes and proteins that are affected by CB101 will be identified. The genes and products (e.g., mRNA transcripts and proteins) will then be used in compound screening to identify inhibitors of these targets; such inhibitors can potentially be and are expected to be additional quinolone potentiators.

### Example 2

### New Synthesis of CB101 and Confirmation of its Activity

A new synthesis of compound 224_C5 (═CB101), was carried out to retest and confirm its inhibitory growth activity in conjunction with norfloxacin (i.e., its ability to potentiate norfloxacin activity).

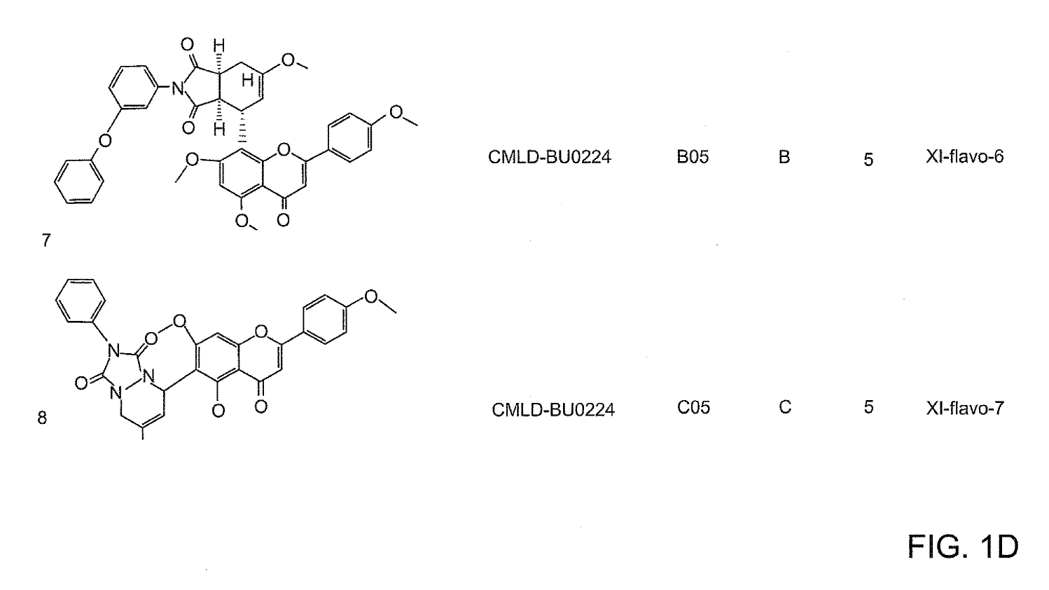

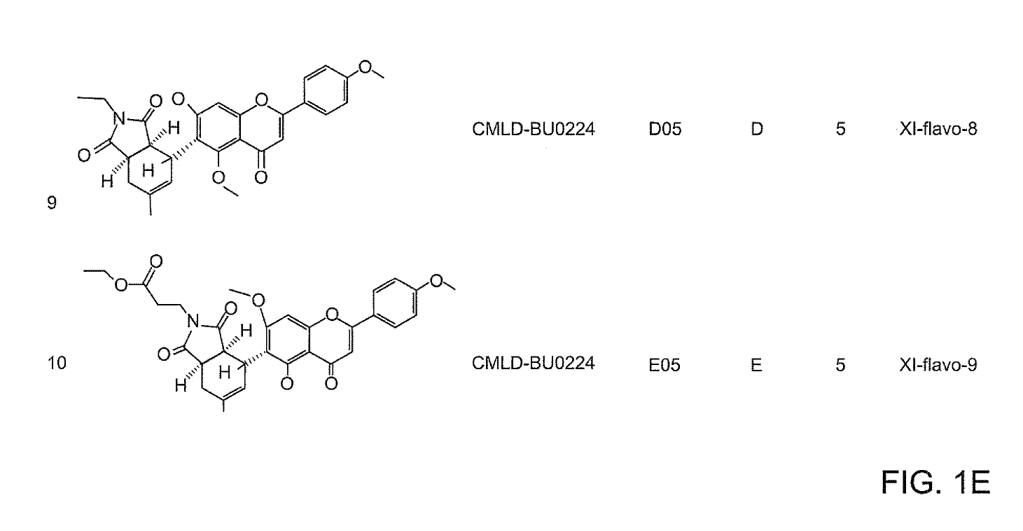

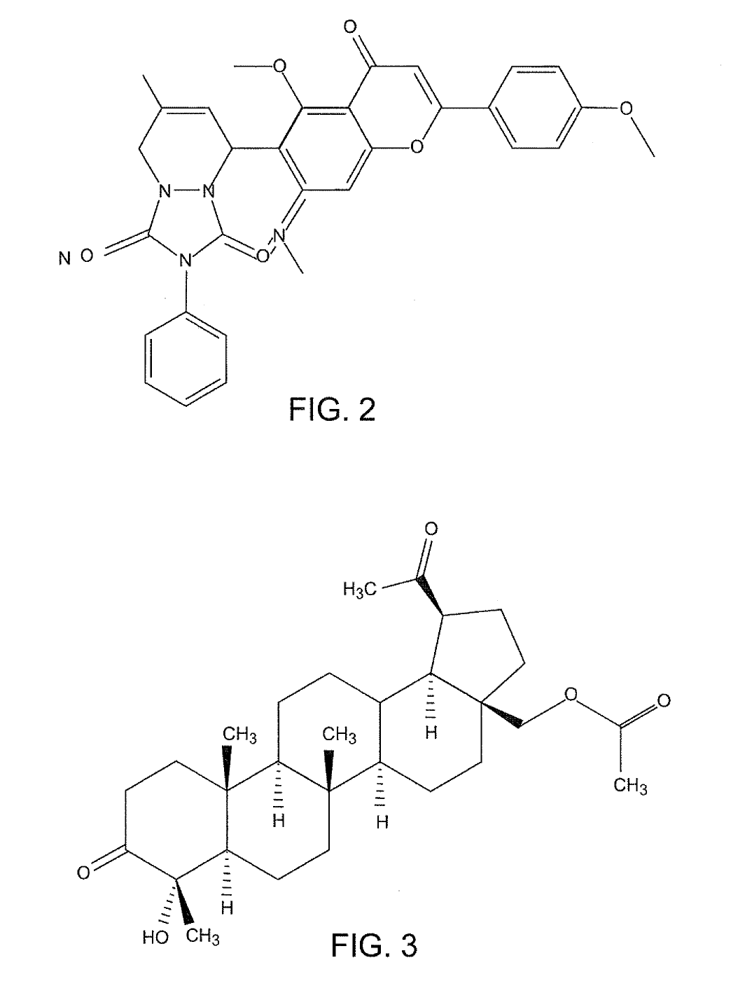

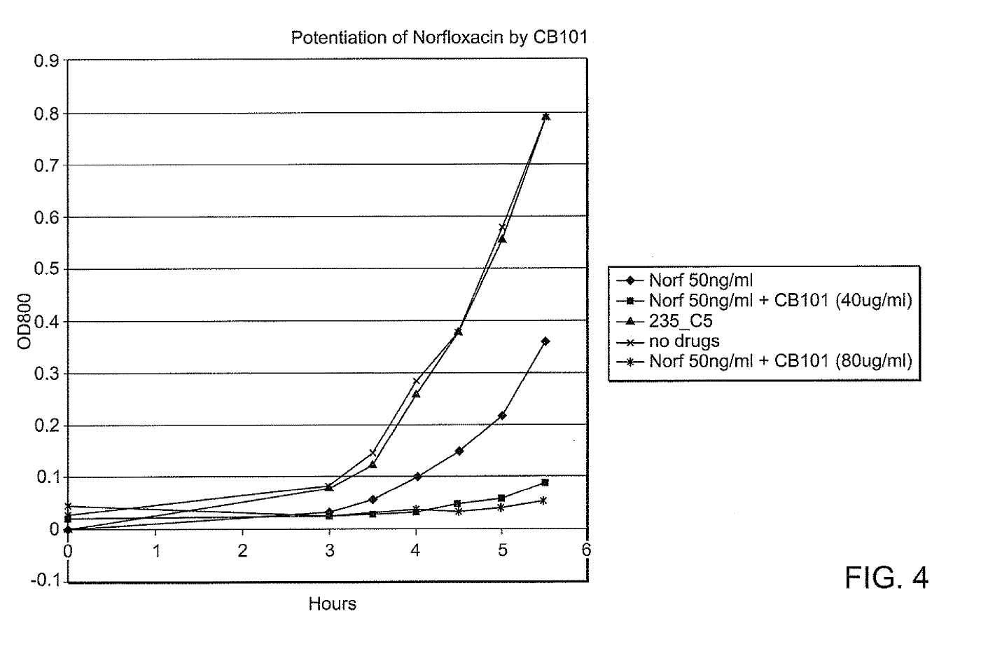

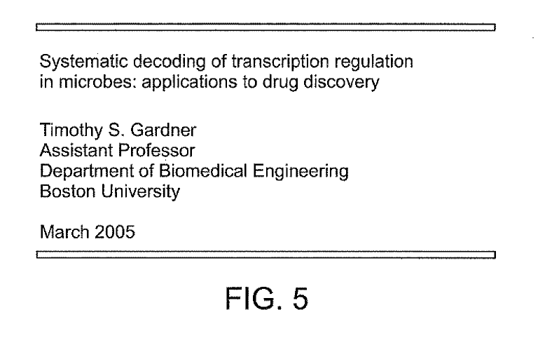

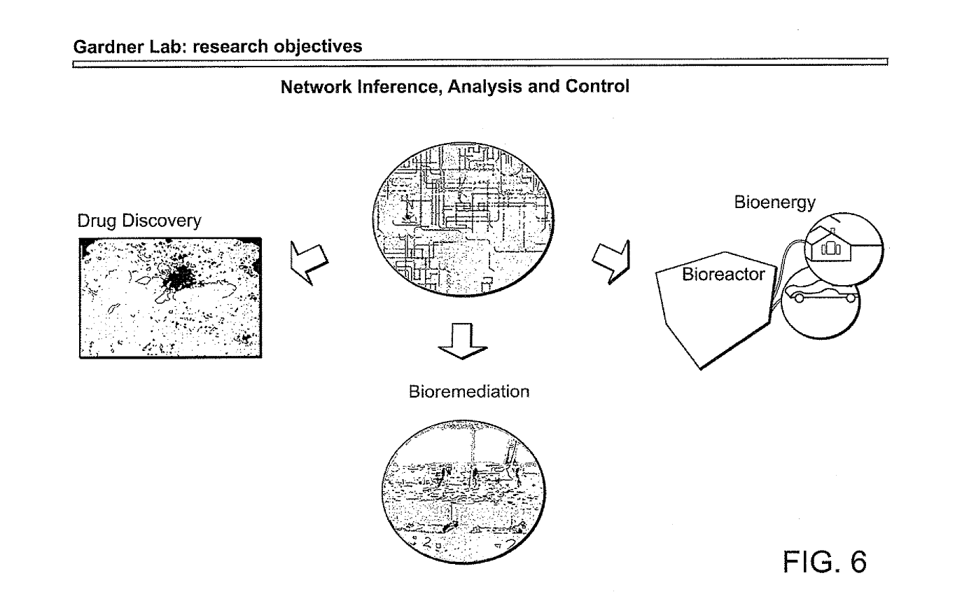

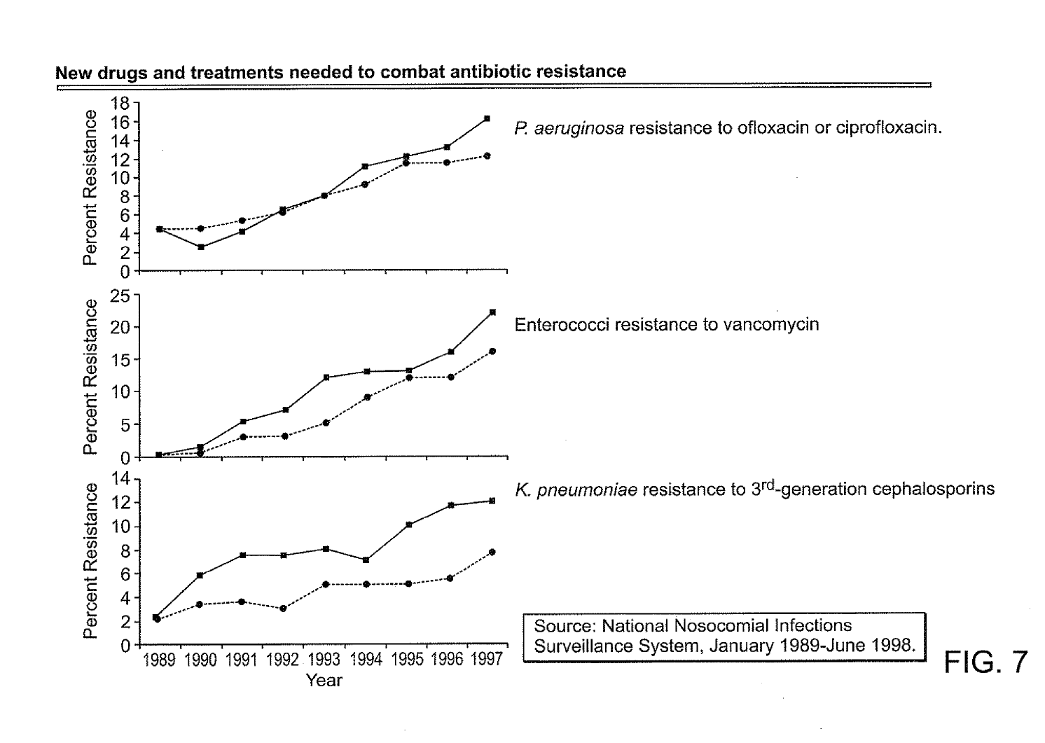

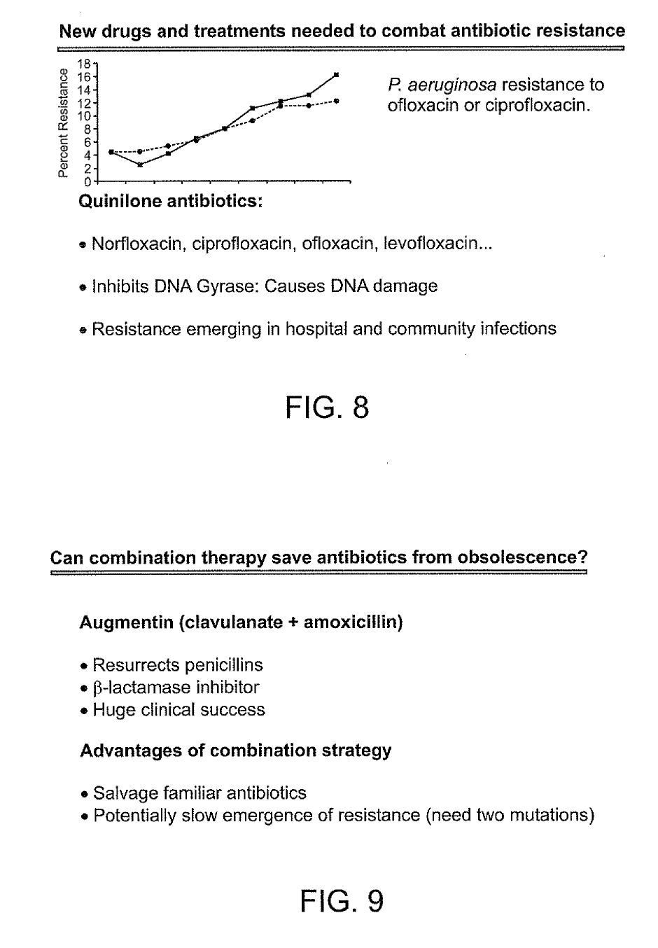

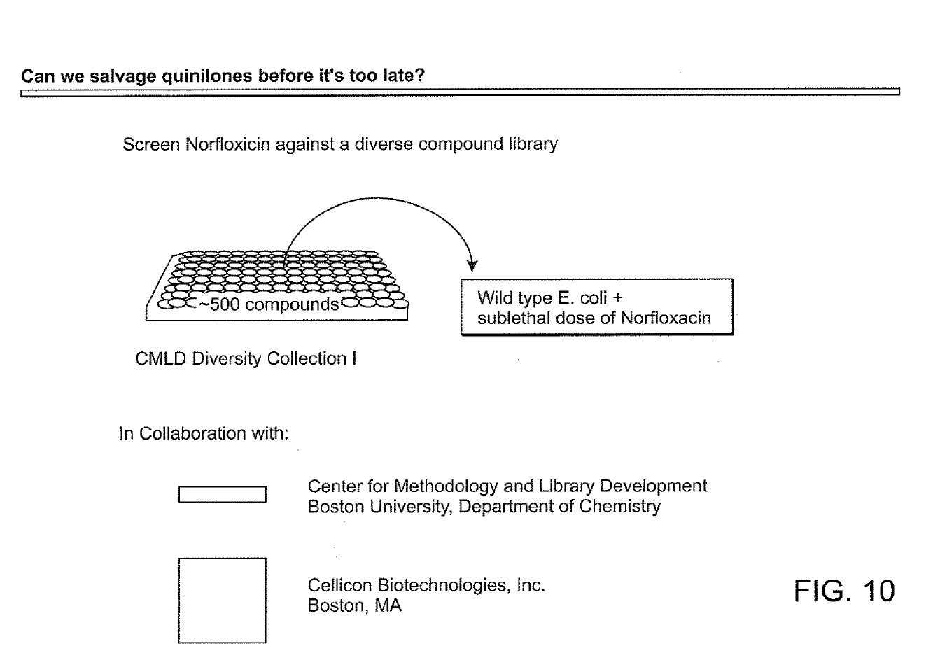

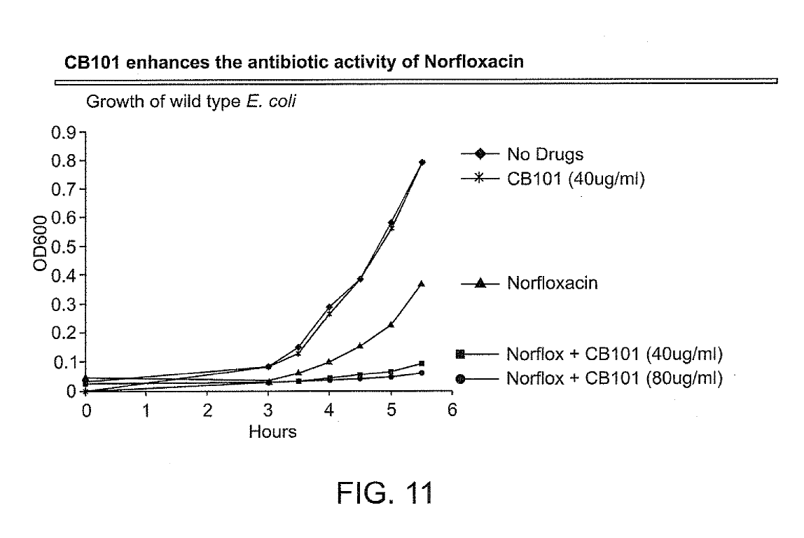

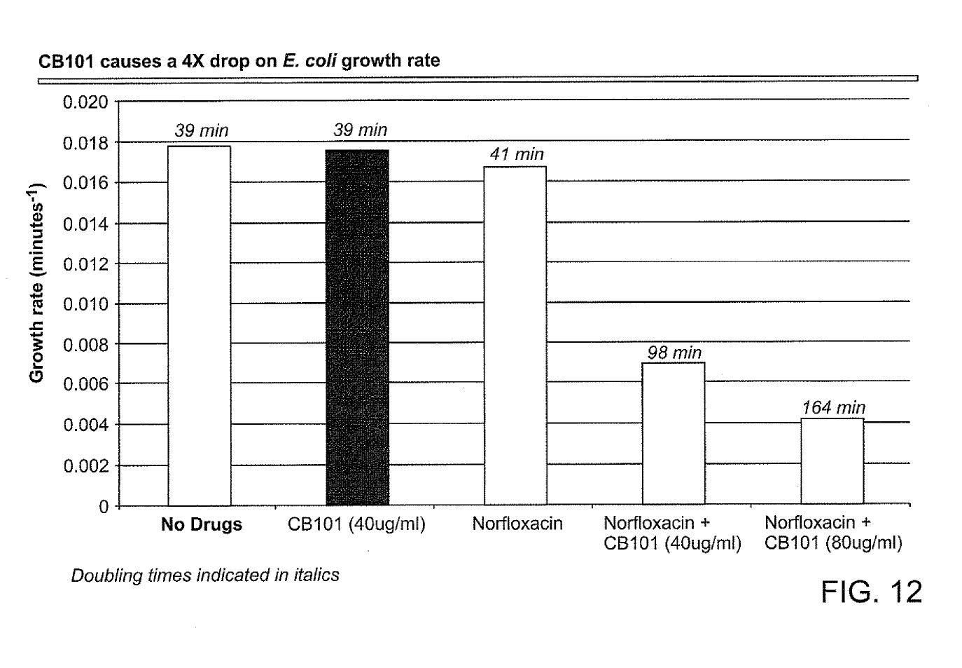

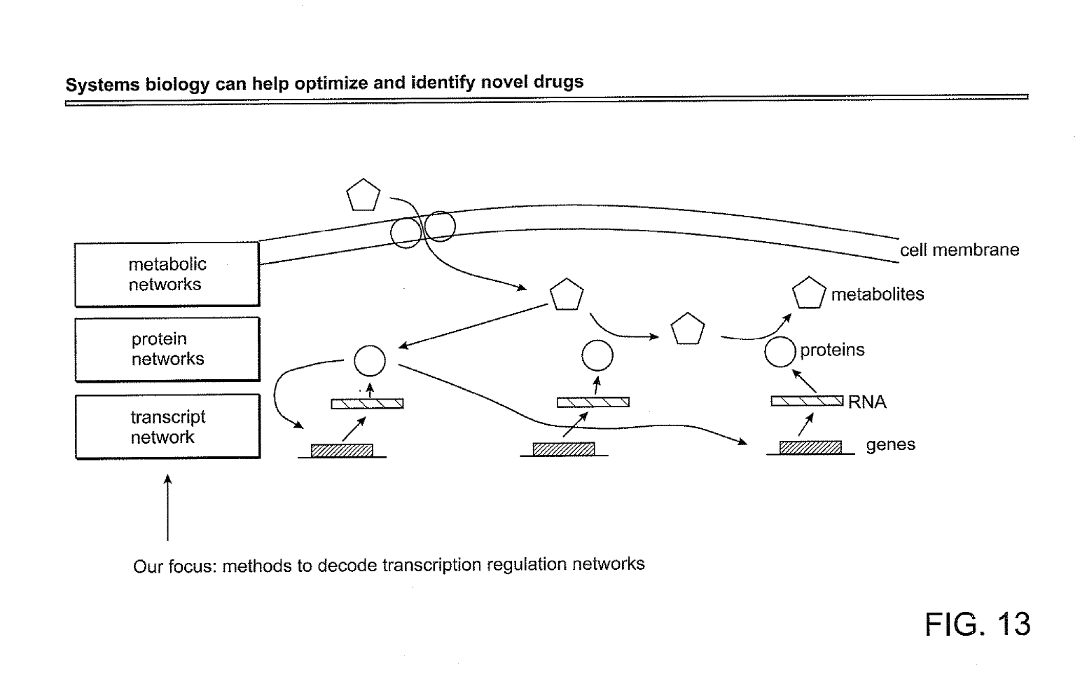

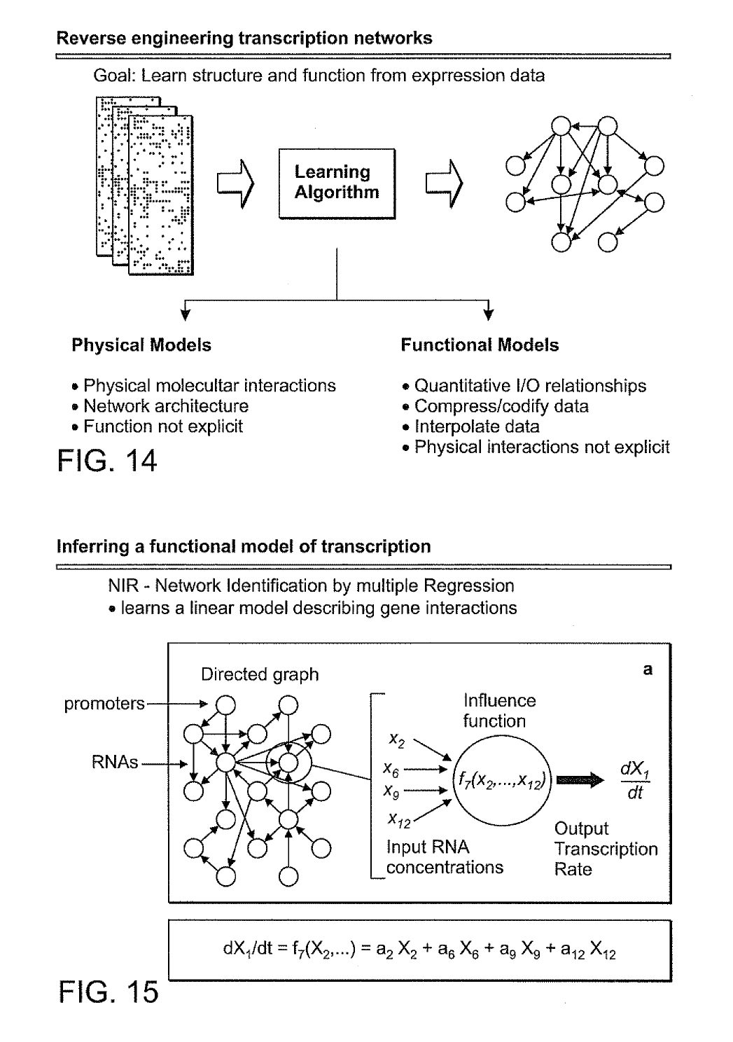

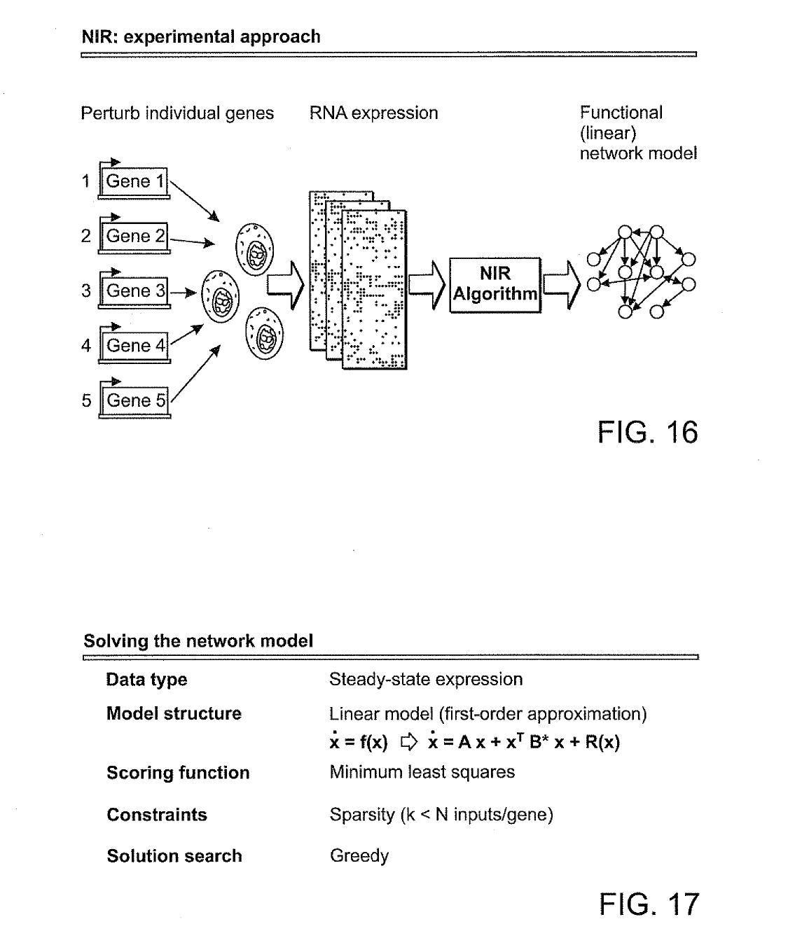

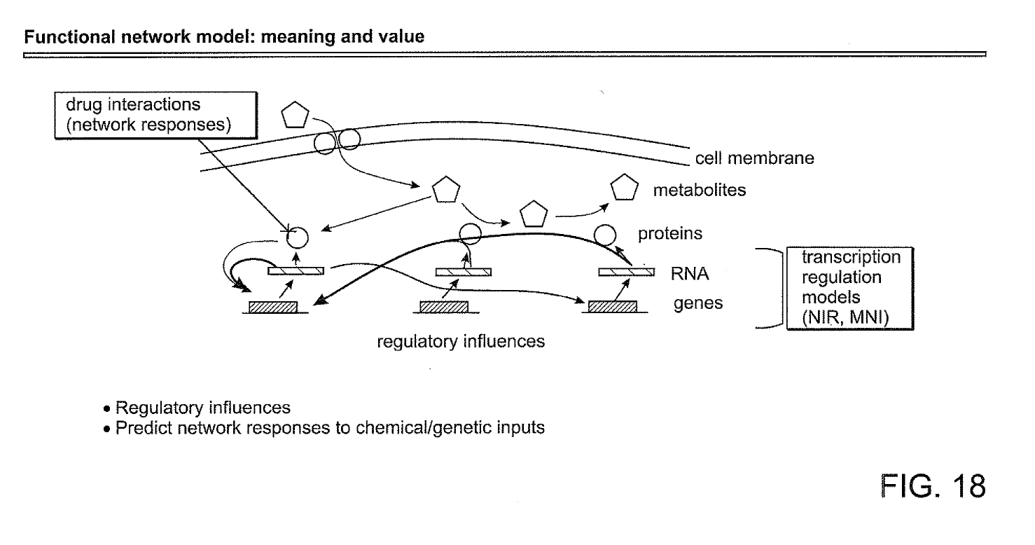

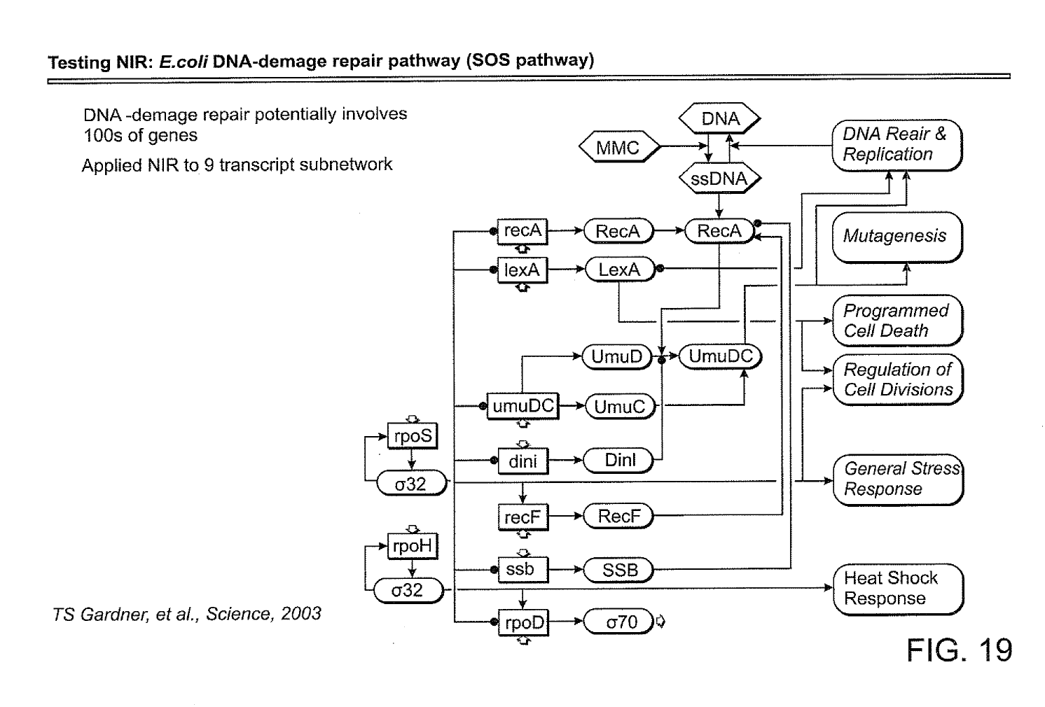

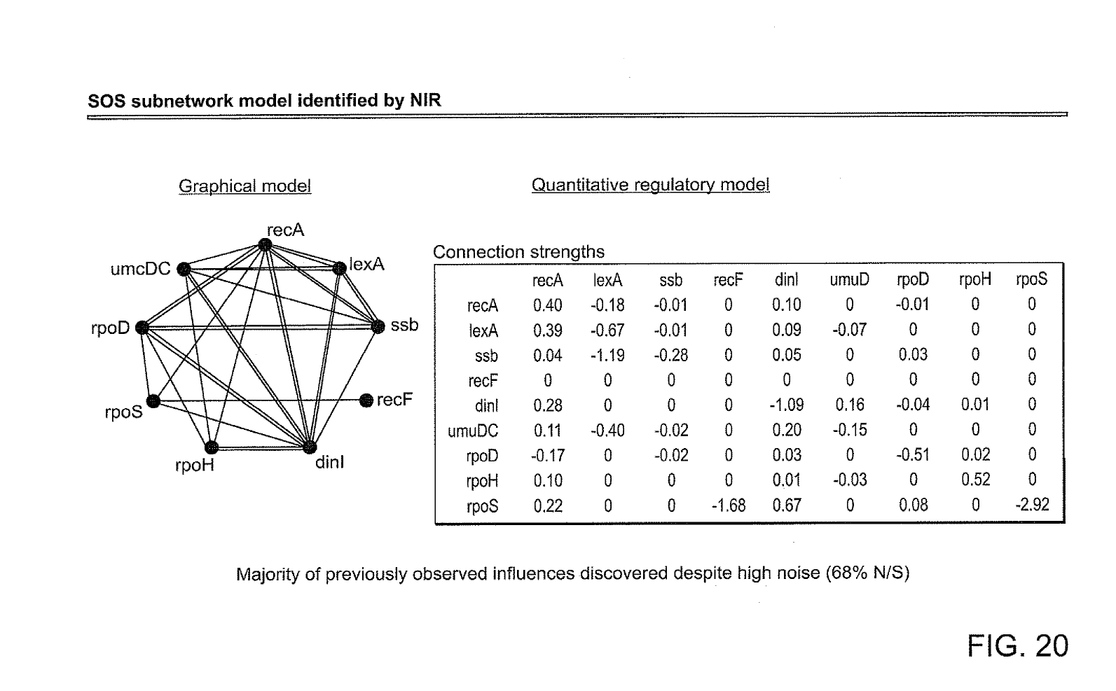

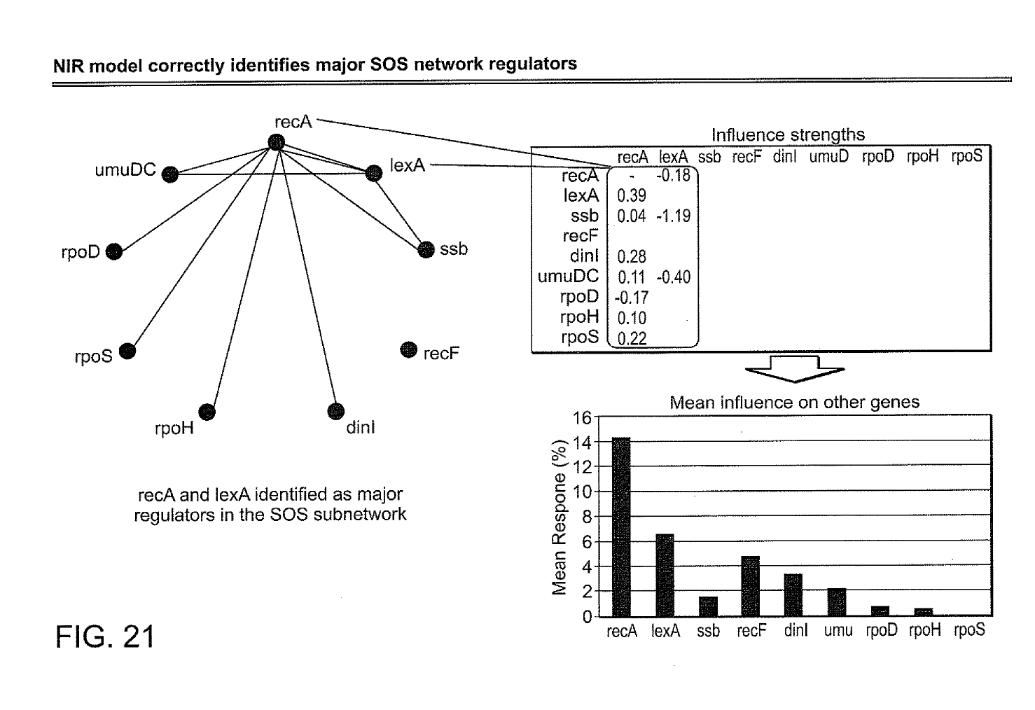

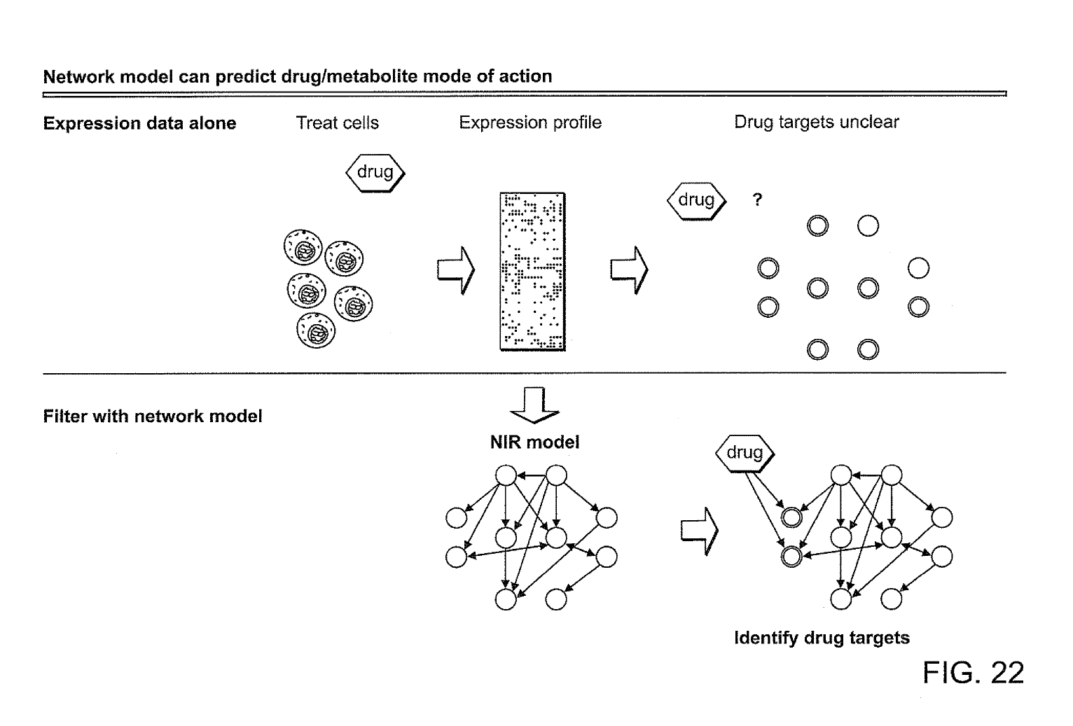

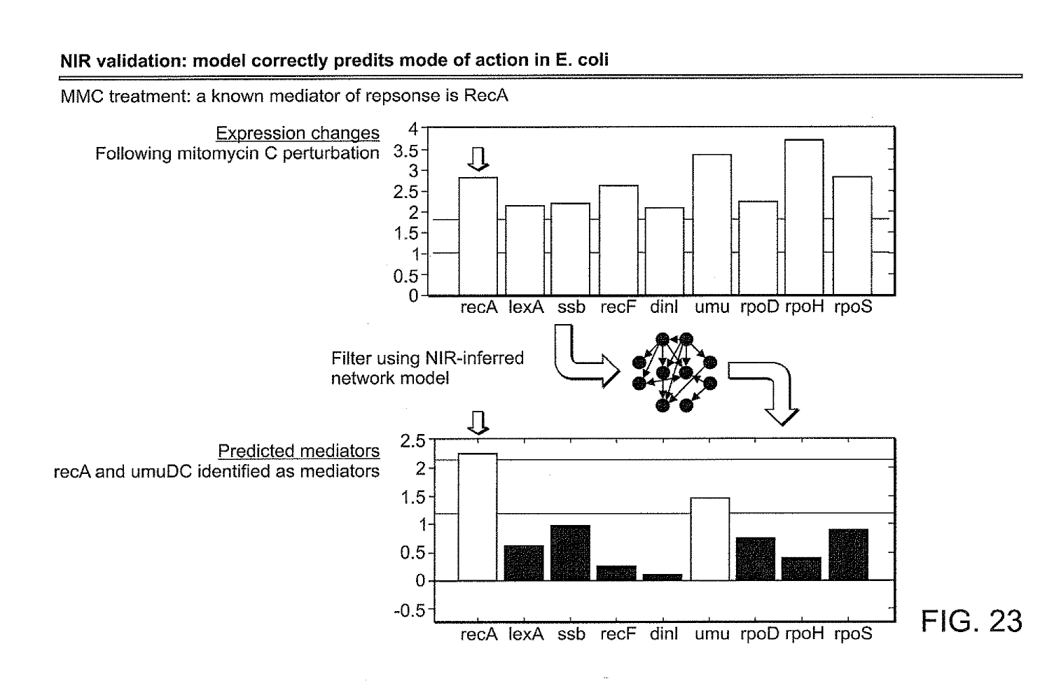

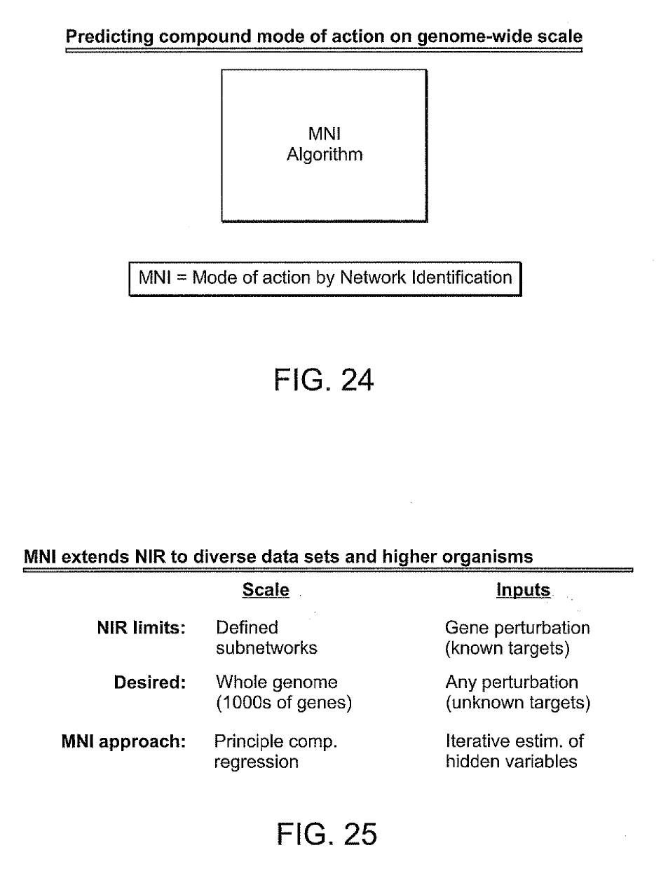

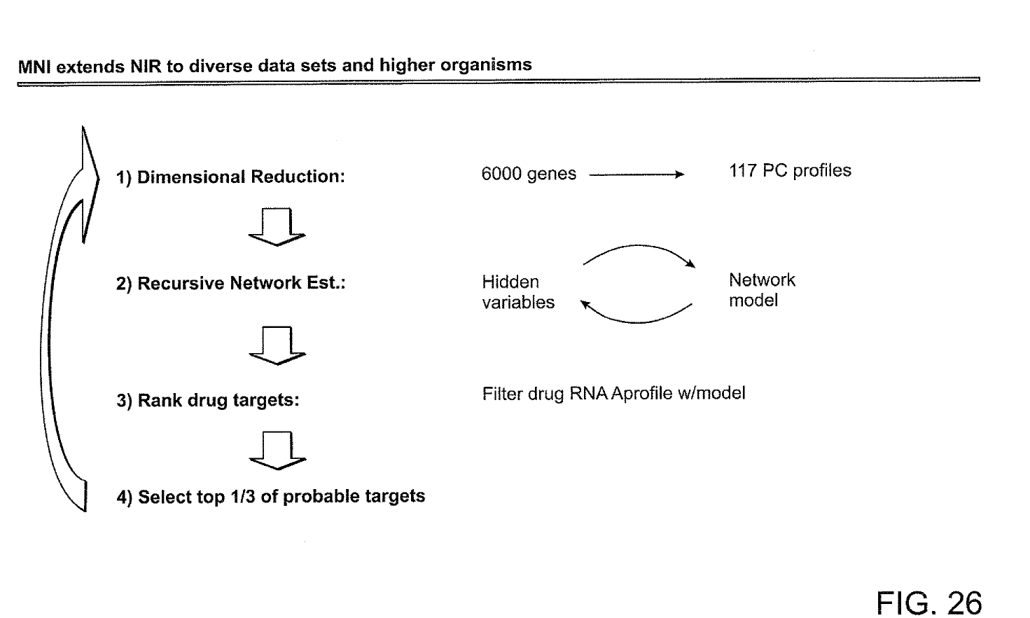

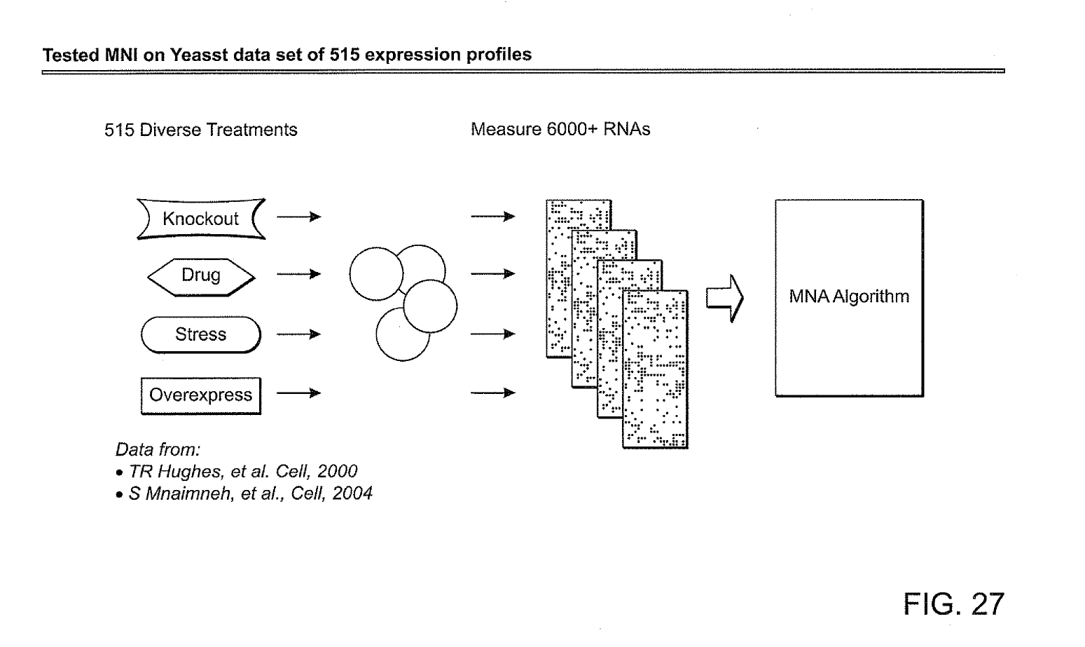

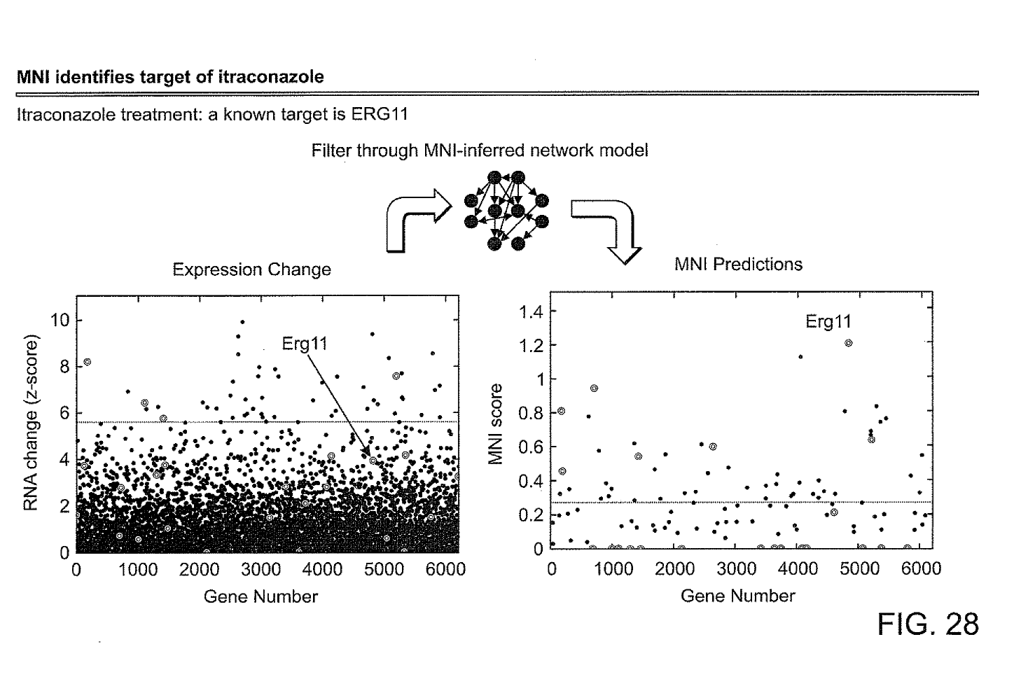

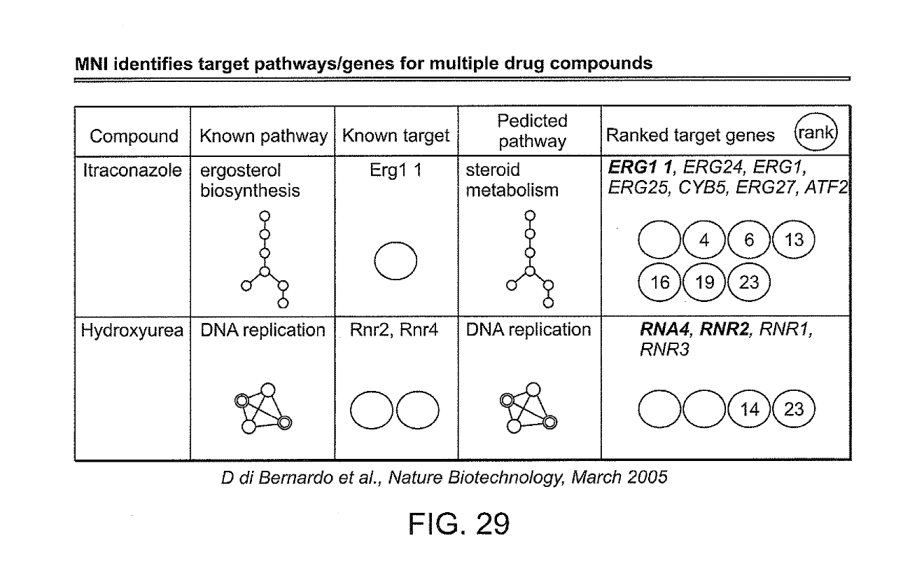

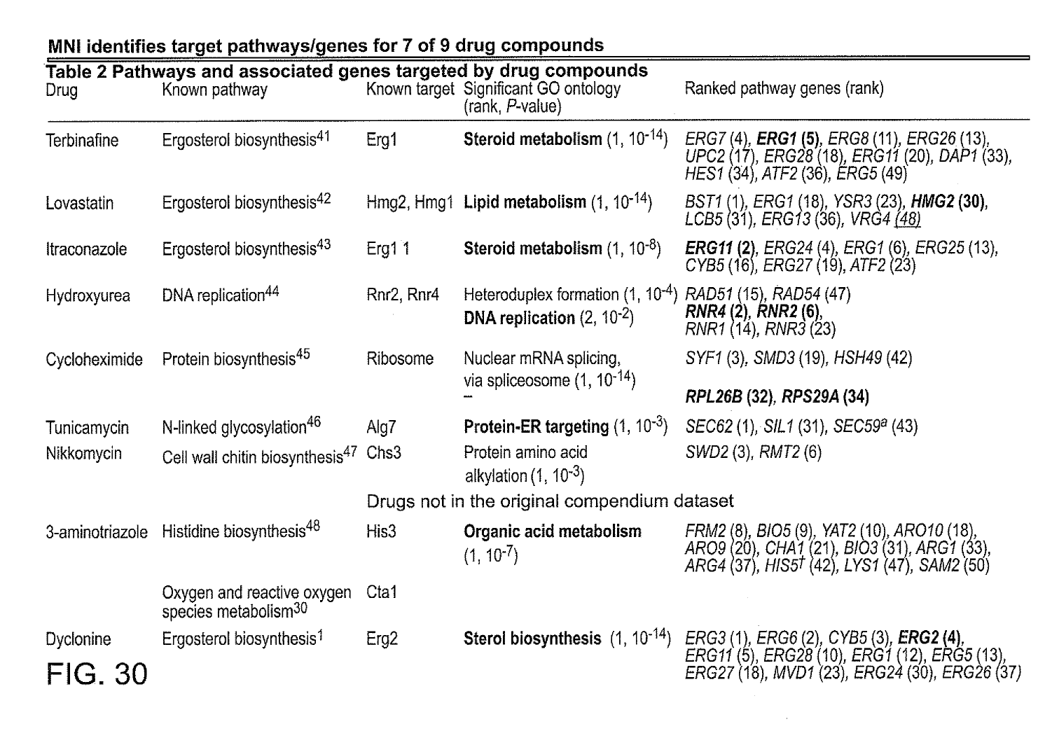

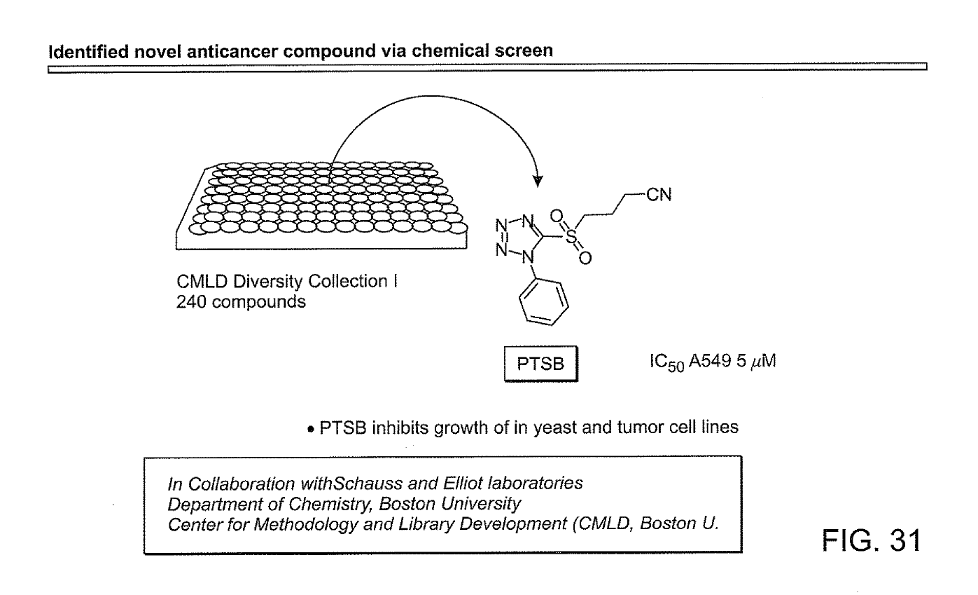

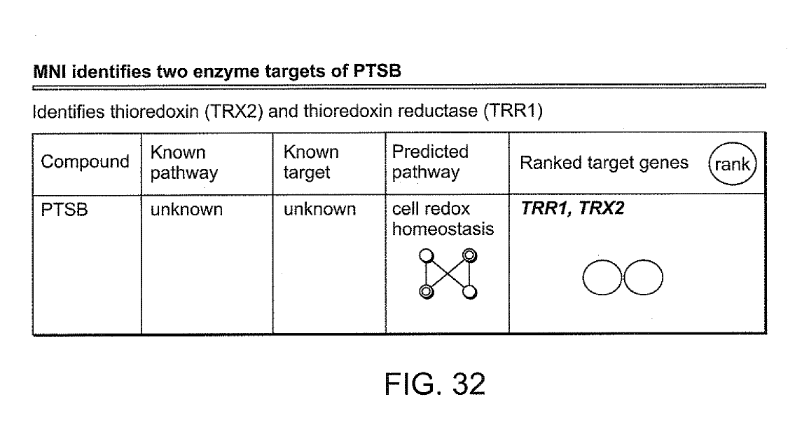

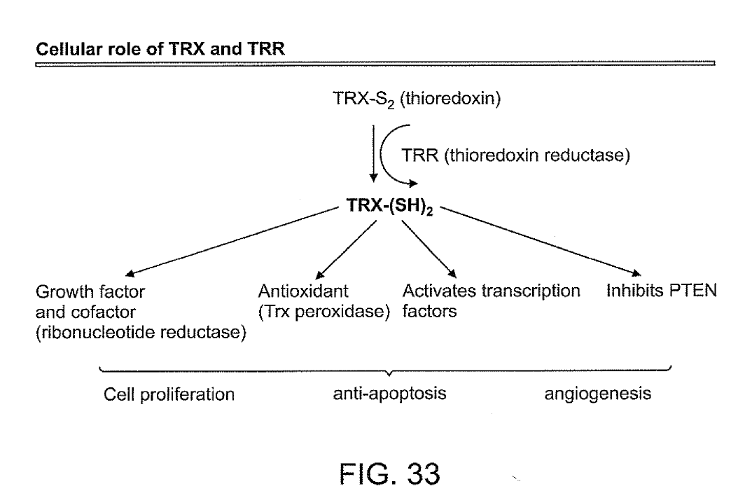

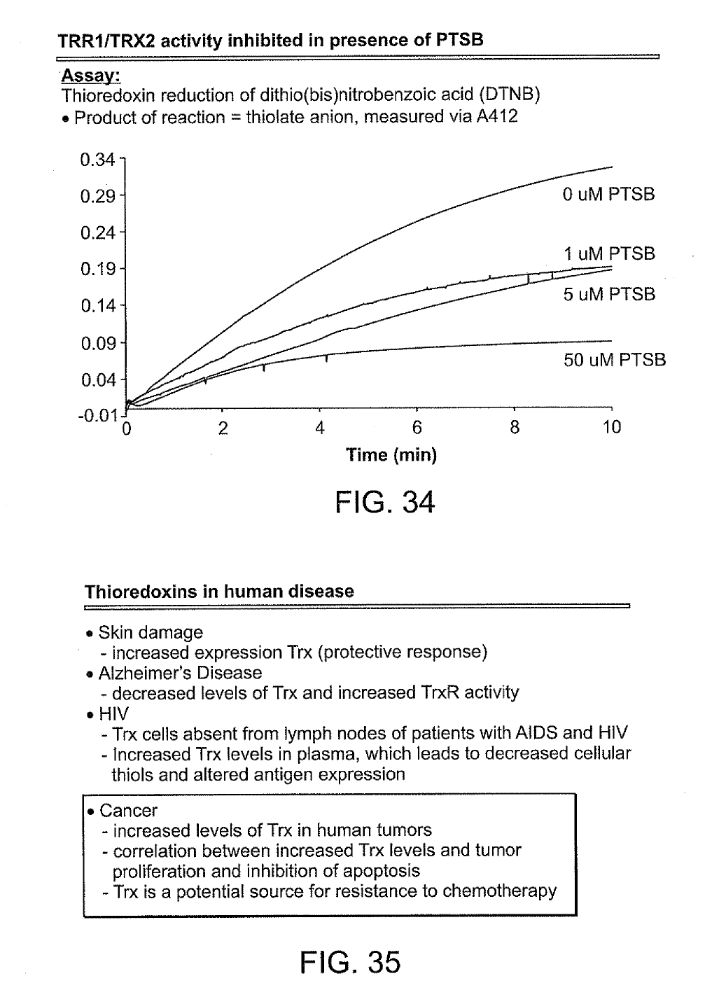

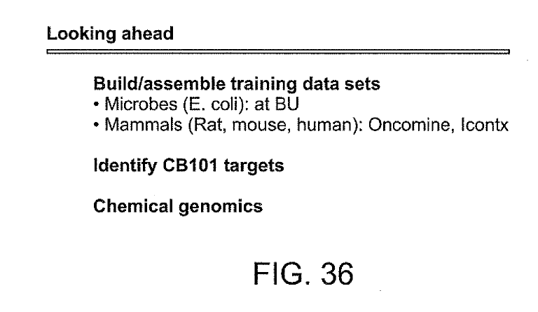

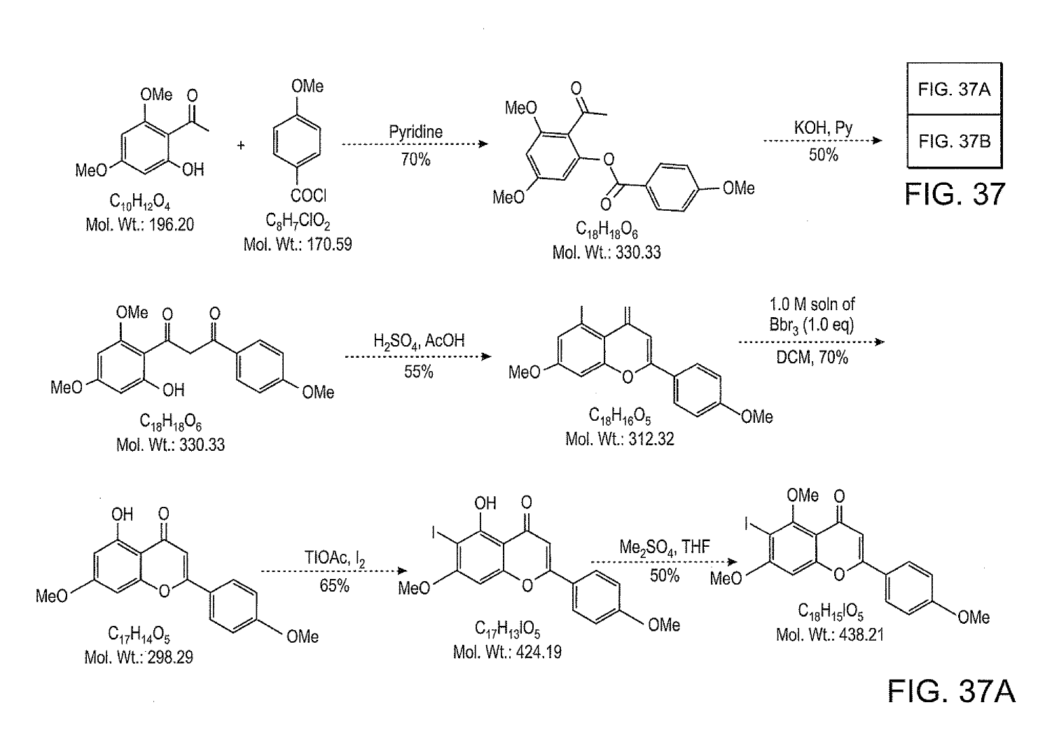

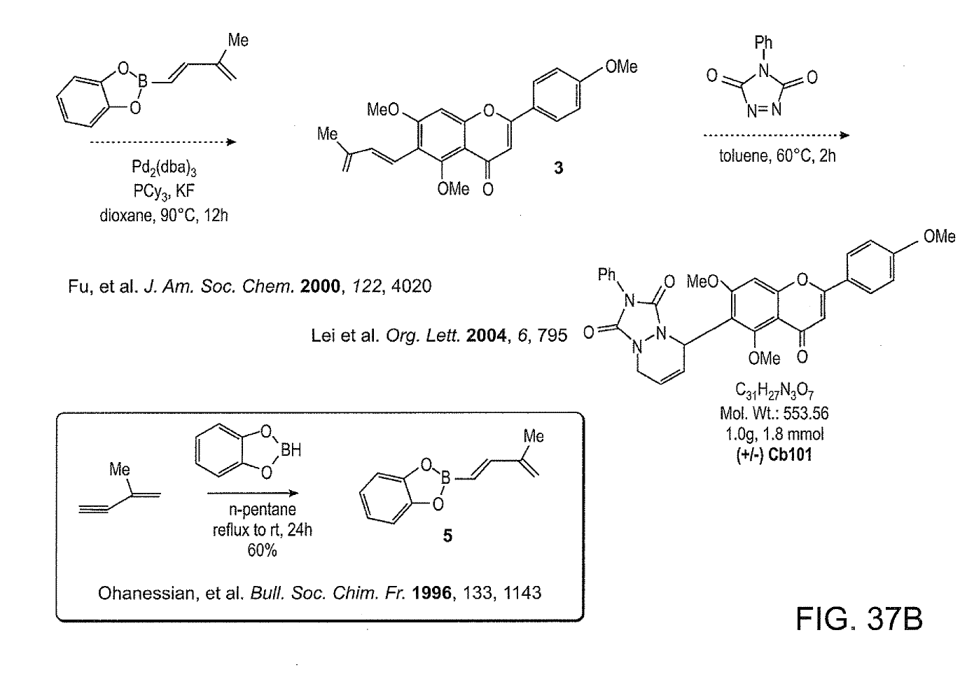

The newly synthesized compound was tested for its ability to potentiate the growth slowing activity in the presence of norfloxacin at 50 ng/mL. The assay was done as described above except that examples were obtained and quantified by OD (600 nm) measurement at different time points. Results are shown in FIG. 4 and confirm the ability of CB101 to potentiate norfloxacin activity. A dose response was observed (i.e., higher concentrations of CB101 resulted in greater potentiating effect). Furthermore, CB101 by itself did not appear to inhibit growth of the test cells or kill them.

### Example 3

### Testing of Activity of CB101 Enantiomers

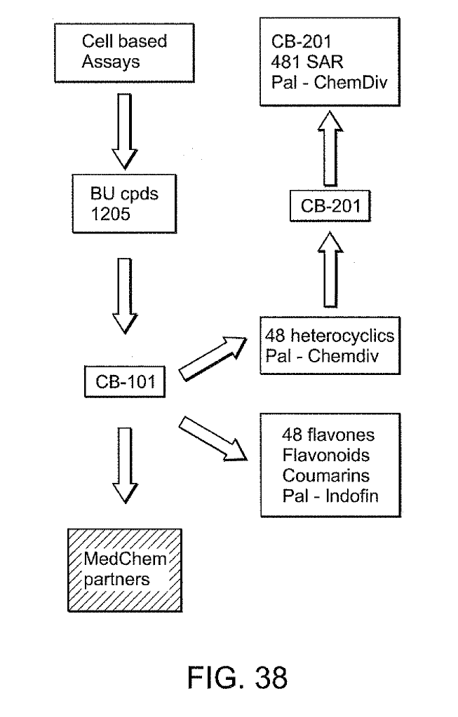

A new synthesis of CB101 (as racemic mixture), carried out as shown on FIG. 37 and described in X. Lei and J. A. Porco Jr., Org. Lett., 2004, 6: 795-798, which is incorporated herein by reference in its entirety, was performed and followed by separation of the enantiomeric (+) and (−) forms by chiral HLPC.

The quinolone resistant strain laboratory S7 was tested for growth in the presence of ciprofloxacin at 0.5 μg/mL (MIC at 1 μg/mL) with 20 μg/mL of CB101 (racemic mixture), the (+) enantiomeric form of CB101 or the (−) enantiomeric form of CB101. No significant differences in potentiating activity were observed between the different enantiomers.

### Example 4

### Screening of “Suggested” Compounds and Identification of Compound CB201

The same assay as that described in Example (i.e., using E. coli, Strain MG1655) was performed on compounds suggested by the Applicants as potentially interesting candidate compounds based on structural similarity to CB101 (see FIG. 38). This screen led to the identification of the compound CB201, the chemical structure of which is presented on FIG. 39.

### Example 4

### Ability of CB101 and CB201 to Potentiate the Activity of Norfloxacin in S. aureus

Cells (S. aureus) were grown in LB in the presence of 0.26 μg/mL of norfloxacin and 5 μg/mL, 10 μg/mL or 20 μg/mL of CB101; or in the presence of 0.25 μg/mL of norfloxacin and 0.625 mg/mL, 1.25 mg/mL or 2.5 mg/mL of CB201; or in the absence of norfloxacin or CB compounds. After 4.5 hours, the cells were washed using PBS and several dilutions were plated on LB plates without the CB compounds. Colony count was measured after overnight incubation. The results of these experiments are presented in FIG. 40 and show that both compounds potentiate norfloxacin in S. aureus. More specifically, both CB101 and CB201 were found to potentiate the quinolone at low concentrations and exhibit anti-growth activity at higher concentrations in S. aureus.

### Example 5

### Ability of CB101 and CB201 to Combat Resistant Clinical Isolates (Staphylococcus isolate S10)

Staphylococcus isolate S10 cells were grown in LB in the presence of 12.5 μg/mL (50% MIC) of ciprofloxacin and 2.5 μg/mL, 5 μg/mL, 10 μg/mL, 20 μg/mL of CB101 or CB201 or in the absence of the CB compounds. Cell density was determined by measuring the optical density at 600 nm during 5 hours of incubation.

The results of these experiments are presented in FIG. 41 and show that both compounds are active at low concentrations (in the lower microgram/mL range) against Staphylococcus clinical isolates resistant to ciprofloxacin.

### Example 6

### In Vivo Validation of Potentiation Activity of CB101

Mice (5 per group) were infect by IP with Staph S7 at an inoculum of 5.225×10⁷ cfu per mouse in a volume of 0.5 mL. Thirty minutes later the animal were injected sub-cutaneously with ciprofloxacin (2, 4, or 6 mg/kg), CB101 (5, 10, or 20 mg) alone or in combination. Animal status (death or not) was recorded 48 hours later.

The results of these experiments are presented in FIG. 42 and FIG. 43 and show that CB101 potentiates ciprofloxacin after S. aureus infection of mice with moderately fluoroquinolone resistant S7 Staphylococcus isolate.

### Example 7

### Serum Binding of CB101 and CB201

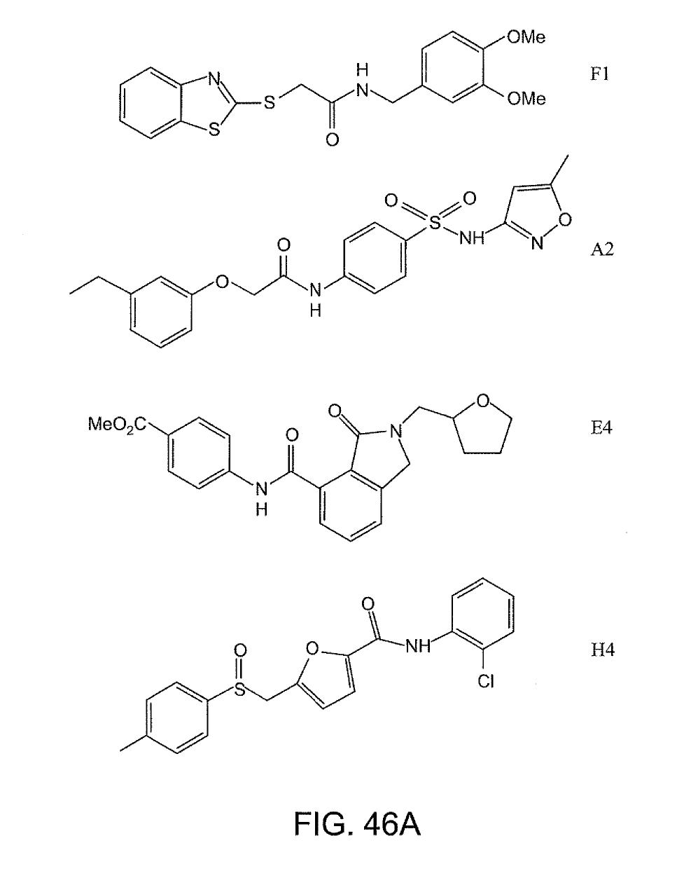

Cells were grown in LB or LB+10% mouse serum with serum and MIC was measured. The results of these experiments are presented on FIG. 44. Change in MIC, which is related to serum binding, was found to be higher for CB101 than for CB201. These results suggest that the presence of serum may reduce activity of these compounds in cellular assays.

### Example 8

### “Suggested” Candidate Compounds Based on CB101

As already mentioned above, CB-101 was the hit identified by the original screening of the Boston University CMLD. The flavone core of this structure is reminiscient of flavopyridol and other kinase inhibitors including genistein and roscovitine. A hypothesis was developed that these compounds may be potential kinase inhibitors, and this hypothesis was further validated by an ATP dependence of RecA for activity. To investigate this hypothesis, a small set of compounds were ordered that are representative of the various different structural motifs that have been reported as kinase antagonists. The selected compounds included 20 flavones, 19 isoflavones, 9 coumarins, and 48 heterocyclic compounds.

These compounds were then screened for quinolone potentiation, as described herein. While none of the isoflavones were found to be active, one flavone, one coumarin, and eight heterocyclic derivatives were found to be active. However a screening hit rate of 10 new ligands from 100 analogs represents a very high rate, and may indicate that the kinase mechanism is indeed valid. The initial flavone lead has now generated at least five different structural motifs that are all active in the quinolone potentiation assay. These new leads represent significant structural departure from CB-101.

The flavone (21150S) and the coumarin (19-281) are shown on FIG. 45. These compounds were purchased from Indofine and are representative of a much larger set of compounds that are commercially available. These compounds could be easily acquired to gain a very rapid understanding of SAR and mechanism of action.

The heterocyclic analogs shown in FIG. 46 were purchased from Chemical Diversity and are representative of a compound collection of over 100,000 discrete structures. The ChemDiv compounds are all combinatorial chemistry based and a large set of analogs are readily available. Rapid SAR could be easily established through an iterative purchasing and screening program. Furthermore, these compounds could also be used to probe the mechanism of action as well as lay the ground work for a provisional patent.

Future plans include the purchase of additional compound from Chemical Diversity to follow up on the screening results of the heterocyclic derivatives. These hits can be categorized in 3-4 general groups and a large number of analogs are readily available which would clarify the SAR around each of these lead molecules.

About 1000-2000 compounds could be purchased to investigate these lead molecules. Several iterations of purchasing analogs (about 500 compounds in each round) and screening would allow a preliminary SAR to be defined and perhaps even potent molecules can be identified in this screening process to validate the therapeutic rationale of RecA as a validated target.

### Example 9

### “Suggested” Candidate Compounds Based on Intermediates in the Synthesis of CB101

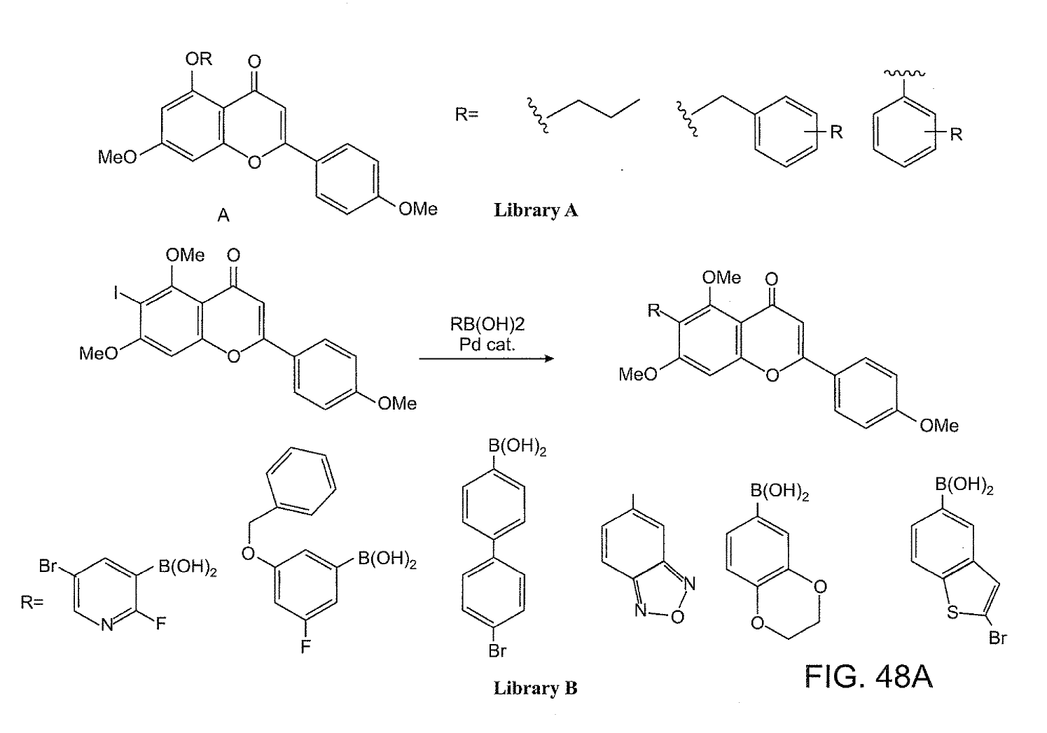

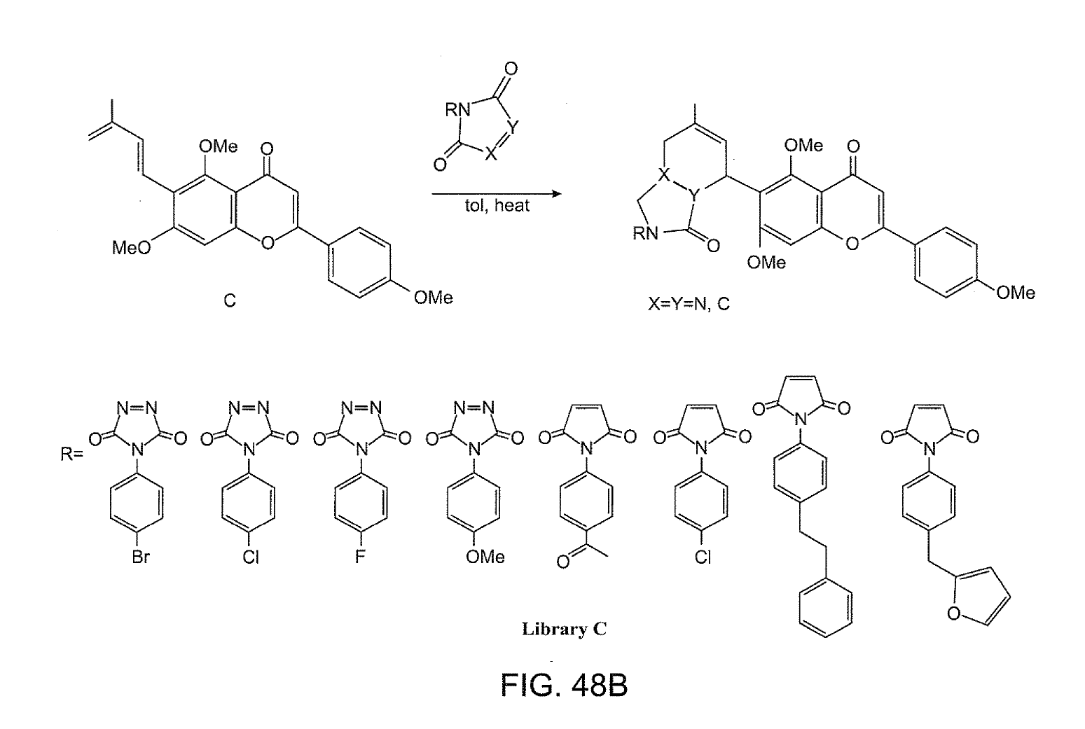

The three intermediates of the synthesis of CB101 are presented on FIG. 47. Three libraries could be developed based on these intermediates (see FIG. 48).

Library A: The only group available for functionalization is the free phenol. 3-4 compounds (ethers) could be synthesized in order to explore the space around this group. The free hydroxyl group may be essential for biological activity; however this will need to be confirmed that at some point to complete the SAR.

Library B: 5-6 compounds could be made, using the Suzuki coupling reaction with substituted boronic acids. The generated structure will be similar to CB-101 and may show some biological activity. The coupling reaction is well precedented and high yielding which will allow to work on a small scale.

Library C: 5-6 compounds could be made using the Diels-Alder reaction of intermediate C with maleimides and azamaleimides and using the reaction conditions developed for the synthesis of CB-101. The synthesized compounds will be direct analogs of CB-101 and will generate SAR about the left-hand side of the molecule.

### Other Embodiments and Equivalents

Other embodiments of the invention will be apparent to those skilled in the art from a consideration of the specification or practice of the invention disclosed herein. It is intended that the specification and examples be considered as exemplary only, with the true scope of the invention being indicated by the following claims.

1. A compound having a structure as shown in any of Formulae 1-10 of FIG. 1, FIG. 2 (CB101), FIG. 3, FIG. 39 (CB201), or any of the compounds of FIG. 48, or a derivative thereof.

2-8. (canceled)

9. A pharmaceutical composition comprising a compound that potentiates quinolone activity and at least one physiologically acceptable carrier.

10. (canceled)

14. The pharmaceutical composition of claim 9, wherein the compound that potentiates quinolone activity is the compound of claim 1, or compound 21150S or compound 19-281 shown in FIG. 45, or a compound having a structure as shown in any of Formulae F1, A2, E4, H4, H5, B5, C6, or E6 of FIG. 46.

15. (canceled)

16. A pharmaceutical composition comprising a quinolone antibiotic, a compound that potentiates the quinolone activity and at least one physiologically acceptable carrier.

17-20. (canceled)

21. The pharmaceutical composition of claim 16, wherein the compound that potentiates the quinolone activity does not have antibiotic activity at concentrations at which it potentiates quinolone activity.

22. The pharmaceutical composition of claim 16, wherein the compound that potentiates the quinolone activity is substantially non-toxic to mammalian cells at concentrations at which it potentiates quinolone activity.

23-26. (canceled)

27. A method of treating a subject in need thereof comprising:
administering a quinolone antibiotic to the subject in combination with a compound that potentiates quinolone activity.

28. The method of claim 27, wherein the compound does not have antibiotic activity at concentrations at which it potentiates quinolone activity.

29-35. (canceled)

36. The method of claim 27, wherein the compound is the compound of claim 1.

37. (canceled)

38. The method of claim 27, wherein the quinolone antibiotic is a fluoroquinolone.

39. (canceled)

40. The method of claim 27, wherein the quinolone antibiotic and the compound are administered in a single composition.

41-42. (canceled)

43. The method of claim 27, wherein the subject is the host of a microorganism, and wherein said microorganism is resistant to the quinoline antibiotic.

44. (canceled)

45. A method of identifying a compound that potentiates activity of an antibiotic, the method comprising steps of:
contacting a cell with an antibiotic agent and a candidate compound; and
identifying the compound as an antibiotic potentiating compound If growth of the cell in the presence of the antibiotic agent and the compound is less than growth of the cell in the presence of the antibiotic agent alone under substantially equivalent conditions.

46-47. (canceled)

48. The method of claim 45, wherein the antibiotic agent is used at a sub-lethal concentration.

49. (canceled)

50. The method of claim 45, wherein the cell is resistant to the antibiotic agent.

51-55. (canceled)

56. The method of claim 45, wherein the method is performed in a high-throughput format.

57. The method of claim 56, wherein two or more compounds are tested simultaneously.

58. (canceled)

59. The method of claim 45 further comprising a step of: performing transcriptional profiling.

60. The method of claim 45 further comprising a step of: identifying a gene, mRNA or protein that is a molecular target of the compound.

61. A computer-readable medium containing results of a screen performed according to the method of claim 45.
